(19) United States
(12) Patent Application Publication
(10) Pub. No.: US 2005/0215529 A1
(43) Pub. Date: Sep. 29, 2005

(54) Method of treating oxidative stress-associated conditions with isopentenyl diphosphate

(75) Inventors: Joseph Holoshitz, Ann Arbor, MI (US); Jie Zheng, Nanjing (CN)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE REGENTS OF THE UNIVERSITY OF MICHIGAN, Ann Arbor, MI (US)

(21) Appl. No.: 11/085,298

(22) Filed: Mar. 21, 2005

Related U.S. Application Data
(60) Provisional application No. 60/555,905, filed on Mar. 24, 2004.

(51) Int. Cl. A61K 31/66

(52) U.S. Cl. 514/102

(57) ABSTRACT

It has been found that isopentenyl diphosphate possesses antioxidant effects 1000 to 10,000 times more potent than classical antioxidants, such as ascorbic acid, β-carotene, and α-tocopherol, and 100 times more potent than 2-chloroadenosine. With such high potency, as disclosed herein, isopentenyl diphosphate can be used to treat or prevent one or more oxidative stress-associated conditions by administering to a host, and preferably a human being, in need thereof a therapeutically effective amount of a pharmaceutical composition containing isopentenyl diphosphate or a pharmaceutically acceptable salt thereof. Additionally, isopentenyl diphosphate can be used to treat or prevent oxidative stress-associated damage to a biomolecule by administering isopentenyl diphosphate or a pharmaceutically acceptable salt thereof to the biomolecule.

### CROSS-REFERENCE TO RELATED APPLICATION

This application claims the benefit under 35 USC § 119(e) of U.S. provisional patent application Ser. No. 60/555,905 filed Mar. 24, 2004, the disclosure of which is incorporated herein by reference.

### STATEMENT REGARDING FEDERALLY SPONSORED RESEARCH

Research relating to the claimed subject matter was supported in part by the United States government under Grant Nos. RO1 A147331, RO1 AR46468, P60 AR20557, and P30 AR48310 awarded by the National Institutes of Health. The government may have certain rights in the claimed subject matter.

### BACKGROUND OF THE INVENTION

1. Field of the Invention

The invention generally relates to methods of treating one or more oxidative stress-associated conditions and, more specifically, to a method of treating a patient afflicted with one or more oxidative stress-associated conditions by administering to the patient a pharmaceutical composition comprising a therapeutically effective amount of isopentenyl diphosphate or a pharmaceutically acceptable salt thereof.

2. Brief Description of Related Technology

Aerobic organisms use oxidative processes, such as oxidative catabolism, metabolism, and phosphorylation, to extract energy from food molecules. These processes provide the organisms with a defense against attacks due to infections or other chemical disturbances. These processes also produce free radicals, such as reactive oxygen species (“ROS”) and reactive nitrogen species.

Oxidative stress—an undesirable imbalance where oxidants outnumber antioxidants—can arise if the rate of ROS production overwhelms existing antioxidant defenses. In such circumstances, a series of cellular responses can occur that can lead to an even greater increase in ROS production. Excessive ROS production, and its otherwise ineffective regulation can be detrimental to cells and tissue, inducing cellular damage that ultimately can lead to cell death (apoptosis). Oxidative stress-associated damage also can cause undesirable changes to the structural and functional integrities of cells that can lead to the propagation of cells instead of apoptosis. Additionally, oxidatively-damaged cellular macromolecules can trigger immune responses that can lead to disease. See generally, D. G. Lindsay et al. (2002) Mol. Aspects of Med. 23:1-38. While oxidative stress may not be responsible for initiating or otherwise causing disease, the progression of the disease can be affected by any resultant oxidative stress. ROS can result from viral and/or bacterial infections, and can be produced by exposure to environmental oxidants, toxicants, and heavy metals, which can disturb the equilibrium between cellular reduction and oxidative reactions and otherwise disrupt normal biological functions.

Optimal control of ROS levels is important for cellular homeostasis, for example. In physiologic concentrations, ROS plays a role in signal transduction events. At excessive concentrations, however, ROS can damage cellular biomolecules, including proteins, lipids, and nucleic acids. ROS has been implicated in the acceleration of cellular senescence, neurodegeneration, malignancy, and atherosclerosis, among other pathologies. Consequently, it has been theorized that the modulation of ROS levels might help prevent, delay the onset of, or even ameliorate these conditions.

Oxidation of DNA can produce a number of molecular alterations, including, for example, cleavage, cross-linkage between DNA and proteins, and oxidation of purines. These alterations are among those generally referred to herein as examples of oxidative DNA damage. Unless corrected by the DNA repair machinery, these alterations can undesirably result in mutations, carcinogenesis, and senescence. Such alterations also are responsible for the oxidative stress that is believed to be responsible for pathogenesis of many neurological and age-associated diseases, such as, for example, atherosclerosis, autoimmune diseases, cancer, cardiovascular disease, cataract, dementia, diabetes and diabetic vasculopathy, and neurodegenerative diseases. A tightly-regulated network of intracellular mechanisms has evolved to protect and ensure the genomic stability and to address oxidative stress. Among the more prominent intracellular mechanisms responsible for modulating oxidative stress are the thioredoxin system, and the antioxidant enzymes catalase, glutathione peroxidase and superoxide dismutase.

In addition to the intracellular mechanisms mentioned above, oxidative stress can be modulated by exogenous ligands that activate the cyclic AMP (cAMP)-dependent protein kinase (PKA) pathway and by low molecular weight antioxidant compounds. Elevated [cAMP]ᵢ blocks many biological effects of hydrogen peroxide (H₂O₂), including filamin redistribution and increased permeability in endothelial cells, P-glycoprotein downregulation in prostate cancer cells, neutrophil adherence to human umbilical vein endothelial cells (HUVEC), and C-Jun N-terminal kinase activation in Chinese hamster V79 cells. Additionally, extracellular adenosine inhibits oxidative burst in neutrophils and protects against ischemia-reperfusion renal injury through A2a-mediated [cAMP]ᵢ increase.

Low molecular weight antioxidants include selenium and phytochemicals, such as ascorbic acid (vitamin C), α-tocopherol (vitamin E), β-carotene, and derivatives thereof, as well as a chemically-less characterized assortment of plant-derived antioxidants and food supplements. For example, β-carotene is capable of quenching a singlet oxygen and has been shown to exert an antioxidant effect in vitro. K. Fukuzawa et al. (1998) Biofactors 7:31-40. Another isoprenoid, ubiquinone (coenzyme Q10) is an electron carrier in the inner mitochondrial membrane. In its reduced form, ubiquinone has been shown to protect lipids, proteins, and DNA against oxidative damage. H. Nohl et al. (1998) Ann. NY Acad. Sci. 854:394-409. Polyisoprenyl diphosphates, which also are isoprenoids, exert antioxidant effects and, at nanomolar concentrations, inhibit phospholipase D and superoxide generation in human neutrophils. See B. Levy et al. (1997) Nature 389:985-990; B. Levy et al (2002) Cell. Mol Life Sci. 59:729-741. Polyisoprenyl monophosphates, however, do not exert a similar antioxidant effect at equimolar concentrations. Yet another class of isoprenoids, prenylated proteins, and specifically the heterotrimeric G proteins and the small GTP-binding proteins (i.e., Ras, Rac, and Rap), whose proper membrane localization and activation state are dependent upon isoprenylation, have been shown to function as ROS and RNS regulators. See M. Santillo et al. (1996) Biochem. Biophysic. Res. Comm. 229:739-745; M. Sundaresan et al. (1996) Biochem. J. 318:379-382; H. Chen et al. (2000) Hypertension 36:923-928.

The clinical effectiveness of these compounds, however, is a matter of on-going debate due in-part to the dual antioxidant and pro-oxidant effects of many of these agents, such as ascorbic acid and β-carotene, for example, and also due in-part to high minimum inhibitory concentrations (IC₅₀ values) necessary for producing a meaningful antioxidant effect. For example, each of the three chemically-characterized antioxidants (i.e., ascorbic acid, α-tocopherol, and β-carotene) and derivatives thereof requires high concentrations in the nanomolar to micromolar-range to exert any meaningful antioxidant effect. Such high concentrations, of course, implicate toxicity issues. Thus, while, many of these antioxidants are believed to decrease mutation rates, the therapeutic utility of each remains highly uncertain.

In view of the foregoing, it would be desirable to find an agent capable of preventing, delaying the onset of, or even ameliorating oxidative DNA damage, cellular senescence, neurodegeneration, malignancy, and atherosclerosis, and other pathologies in a manner heretofore unattainable with conventional antioxidant agents. Furthermore, it would be desirable to find an agent capable of treating, preventing, delaying the onset of, and/or otherwise ameliorating the symptoms of oxidative stress-associated diseases.

### SUMMARY OF THE INVENTION

The present invention addresses a need in the art to provide an agent and methods of treating, preventing, delaying the onset of, and/or otherwise ameliorating the symptoms of oxidative stress-associated conditions. Accordingly, disclosed herein is a method of treating one or more oxidative stress-associated conditions. The method includes administering to a host (preferably a mammal) in need thereof a therapeutically effective amount of a pharmaceutical composition containing isopentenyl diphosphate or a pharmaceutically acceptable salt thereof. The pharmaceutical composition also can include a pharmaceutically acceptable carrier and one or more optional cosmetic and pharmaceutical ingredients commonly used in the skin care industry, for example.

In one embodiment, a method of preventing one or more oxidative stress-associated conditions includes administering to a host (preferably a mammal) in need thereof a therapeutically effective amount of a pharmaceutical composition containing isopentenyl diphosphate or a pharmaceutically acceptable salt thereof.

In another embodiment, an article of manufacture includes a packaged pharmaceutical composition containing isopentenyl diphosphate or a pharmaceutically acceptable salt thereof. The article also includes an insert providing instructions for administration of the composition to treat or prevent an oxidative stress-associated condition in a host (preferably a mammal), and a container for the packaged pharmaceutical composition and the insert.

In yet another embodiment, a method of decreasing the oxidative stress level in a cell (preferably a mammalian cell) includes contacting the cell with isopentenyl diphosphate or a pharmaceutically acceptable salt thereof under conditions effective to decrease the level of an oxidizing species present in the cell in response to an oxidative stress compared to the same conditions when the isopentenyl diphosphate or a pharmaceutically acceptable salt thereof is not present.

In a further embodiment, a method of decreasing the oxidative stress level in a host (preferably a mammal) includes administering to the host isopentenyl diphosphate or a pharmaceutically acceptable salt thereof under conditions effective to decrease the level of an oxidizing species present in the host in response to an oxidative stress compared to the same conditions when the isopentenyl diphosphate or a pharmaceutically acceptable salt thereof is not present.

In a still further embodiment, a method of treating a biomolecule damaged by oxidative stress includes administering isopentenyl diphosphate or a pharmaceutically acceptable salt thereof to the biomolecule. Preferably, the biomolecule is selected from the group consisting of proteins, lipids, and nucleic acids.

In another embodiment, a method of preventing oxidative stress-associated damage to a biomolecule includes administering isopentenyl diphosphate or a pharmaceutically acceptable salt thereof to the biomolecule. Preferably, the biomolecule is selected from the group consisting of proteins, lipids, and nucleic acids.

Additional features of the invention may become apparent to those skilled in the art from a review of the following detailed description, taken in conjunction with the drawing figures, the examples, and the appended claims.

### BRIEF DESCRIPTION OF THE DRAWING FIGURES

For a more complete understanding of the invention, reference should be made to the following detailed description and accompanying drawings wherein:

FIG. 1 illustrates a suitable biosynthetic pathway, specifically a 1-deoxy-D-xylulose-5-phosphate (“DOXP”) pathway, to obtain isopentenyl diphosphate;

FIG. 2 illustrates another suitable biosynthetic pathway, specifically a mevalonate (“MVA”) pathway, to obtain isopentenyl diphosphate;

FIG. 3 graphically illustrates ROS generation as a function of the molar concentration of isopentenyl diphosphate;

FIG. 4 graphically illustrates the levels of ROS generation in human fibroblastoid M1 cells treated with and without 10 μM 2-chloroadenosine (“2CA”) or 10 μM isopentenyl diphosphate (“IPP”);

FIG. 5 shows fluorescence photomicrographs of representative human fibroblastoid M1 cells subjected to the “Comet” assay;

FIG. 6 graphically illustrates DNA damage protection as a function of the concentration of 2-chloroadenosine;

FIG. 7 graphically illustrates DNA damage protection as a function of the concentration of isopentenyl diphosphate;

FIG. 8 graphically illustrates DNA damage protection of A2aAR-transfected human embryonic kidney 293 (“HEK293”) cells by 2-chloroadenosine;

FIG. 9 graphically illustrates DNA damage protection of A2bAR-transfected HEK293 cells by 2-chloroadenosine;

FIG. 10 graphically illustrates DNA damage protection of A1AR-transfected HEK293 cells by 2-chloroadenosine and cyclic AMP;

FIGS. 11-13 are graphs illustrating the DNA damage protection as a function of the molar concentration of Prostaglandin E1 (“PGE,”) (FIG. 11), forskolin (“FSK”) (FIG. 12), and 8-bromo adenosine 3′,5′-cyclic monophosphate (“8-Br-cAMP”) (FIG. 13);

FIGS. 14-16 are graphs illustrating the DNA damage protection as a function of the molar concentration of H-89 (FIG. 14), 8-bromoguanosine-3′,5′-cyclic monophosphate (“8-Br-cGMP”) (FIG. 15), and SNAP (FIG. 16);

FIG. 17 graphically illustrates DNA damage protection of A1AR-transfected HEK293 cells by isopentenyl diphosphate;

FIG. 18 graphically illustrates the effect of 2-chloroadenosine and isopentenyl diphosphate on c[AMP]ᵢ as a function of time;

FIGS. 19A and 19B graphically illustrate that, unlike 2CA-mediated DNA damage protection, the IPP-mediated effect is resistant to the A2bAR antagonist, enprofylline;

FIG. 20 graphically illustrates that, unlike 2CA-mediated DNA damage protection, the IPP-mediated effect is resistant to the PKA inhibitor, H-89;

FIG. 21 graphically illustrates that antioxidative signaling by isopentenyl diphosphate—but not by 2-chloroadenosine—is dependent upon the proteasome-system;

FIG. 22 graphically illustrates that IPP-mediated DNA damage protection signaling—but, not 2CA-mediated DNA damage protection signaling—is dependent upon the mevalonate pathway;

FIG. 23A graphically illustrates DNA damage protection obtainable with farnesyl diphosphate (“FP P”) or geranylgeranyl diphosphate (“GG PP”) as a function of the concentration of isoprenyl;

FIG. 23B graphically illustrates DNA damage protection obtainable with isopentenyl diphosphate (“IPP”), or isoprenyl monophosphate (“IMP”) as a function of the concentration of isoprenyl;

FIGS. 24A and 24B graphically illustrate that incubation of human fibroblastoid M1 cells with a geranylgeranyl transferase (“GGT”) inhibitor effectively blocked IPP-mediated DNA damage protection, whereas it has no appreciable affect on 2CA-mediated DNA damage protection signaling; and,

FIG. 25 graphically illustrates that incubation of human fibroblastoid M1 cells with a farnesyl transferase (“FT”) inhibitor had no effect on either IPP-mediated DNA damage protection signaling nor on 2CA-mediated DNA damage protection signaling.

While the disclosed invention is susceptible of embodiments in various forms, there are illustrated in the drawings (and will hereafter be described) specific embodiments of the invention, with the understanding that the disclosure and drawings are intended to be illustrative, and are not intended to limit the invention to the specific embodiments described herein.

### DETAILED DESCRIPTION OF THE INVENTION

Based upon its antioxidant effects, β-carotene is considered to be an agent effective at decreasing mutation rates, though its therapeutic utility remains in doubt. Carotenoids, such as β-carotene, are generated in plants via a mevalonate pathway or a 1-deoxyxylulose-5-phosphate pathway. Like all isoprenoids, the biosynthesis of carotenoids involves successive steps of chain-elongation using isopentenyl diphosphate as a building block. The ability to regulate oxidative stresses is shared by several members of the isoprenoid family, including β-carotene, ubiquinone, polyisoprenyl diphosphates, and prenylated proteins. Heretofore, however, building blocks and/or intermediates in the isoprenoid biosynthetic pathways have not been considered or found to share a similar ability to regulate oxidative stresses. It has now been found that exogenously added isopentenyl diphosphate is a potent antioxidant with genoprotective activity in the picomolar range (versus 2-chloroadenosine, which is an antioxidant having genoprotective activity in the nanomolar range). While, isopentenyl diphosphate has been shown to trigger signaling events in lymphocytes, heretofore, it has not been shown to possess antioxidant properties. It has been found that isopentenyl diphosphate, as a potent antioxidant, has a 50% inhibitory concentration (IC₅₀) of about 1.7×10⁻¹¹ moles per liter (M). Furthermore, it has been found that the genoprotective effect of isopentenyl diphosphate is mediated through a signaling pathway that is distinct from the pathway by which 2-chloroadenosine operates.

Based in part on this effect and potency, the present invention generally relates to the treatment and prevention of conditions caused by oxidative stress with isopentenyl diphosphate. More specifically, and in one embodiment, the invention is directed to a method of treating one or more oxidative stress-associated conditions with isopentenyl diphosphate. The method includes administering to a host (preferably a mammal) in need thereof a pharmaceutical composition containing a therapeutically effective amount of isopentenyl diphosphate or a pharmaceutically acceptable salt thereof. As the term is used herein, “host” refers to any living organism and, most preferably, it refers to an animal selected from the group consisting of human beings, laboratory animals (e.g., mice, rats, and monkeys), pets, livestock, horses, and zoo specimens. Most preferably, “host” refers to a human being (often referred to herein as the “patient”). While the preferred treated subject is a human being, the invention herein is not limited to such a subject. Indeed, the invention is applicable to biomolecules and other mammalian and non-mammalian subjects for medical research purposes.

In another embodiment, a method of preventing one or more oxidative stress-associated conditions includes administering to a host in need thereof a therapeutically effective amount of a pharmaceutical composition containing isopentenyl diphosphate or a pharmaceutically acceptable salt thereof.

In still another embodiment, an article of manufacture includes a packaged pharmaceutical composition containing isopentenyl diphosphate or a pharmaceutically acceptable salt thereof. The article also includes an insert providing instructions for administration of the composition to treat or prevent an oxidative stress-associated condition in a host, and a container for the packaged pharmaceutical composition and the insert.

In yet another embodiment, a method of decreasing the oxidative stress level in a cell (preferably a mammalian cell) includes contacting the cell with isopentenyl diphosphate or a pharmaceutically acceptable salt thereof under conditions effective to decrease the level of an oxidizing species present in the cell in response to an oxidative stress compared to the same conditions when the isopentenyl diphosphate or a pharmaceutically acceptable salt thereof is not present.

In a further embodiment, a method of decreasing the oxidative stress level in a host includes administering to the host isopentenyl diphosphate or a pharmaceutically acceptable salt thereof under conditions effective to decrease the level of an oxidizing species present in the host in response to an oxidative stress compared to the same conditions when the isopentenyl diphosphate or a pharmaceutically acceptable salt thereof is not present.

In a still further embodiment, a method of treating a biomolecule damaged by oxidative stress includes administering isopentenyl diphosphate or a pharmaceutically acceptable salt thereof to the biomolecule. In another embodiment, a method of preventing oxidative stress-associated damage to a biomolecule includes administering isopentenyl diphosphate or a pharmaceutically acceptable salt thereof to the biomolecule. The subject biomolecule can be in a host, such as a mammal. Thus, the methods can be performed in vitro and in vivo.

The present invention also relates to a pharmaceutical composition containing isopentenyl diphosphate or a pharmaceutically acceptable salt thereof. The pharmaceutical composition also can include a pharmaceutically acceptable carrier and one or more optional cosmetic and pharmaceutical ingredients commonly used in the skin care industry, for example.

Proteins, lipids, and nucleic acids (including DNA) are among the biomolecules that can be damaged by oxidative stress. Oxidative damage to proteins can be brought about by the oxidation of cysteine with formation of disulphide, the oxidation of methionine with formation of sulphoxide and sulphone, and the oxidation of tryptophan and formation of N-formyl kynurenine and kynurenine. Oxidative damage to proteins also can occur by the hydroperoxidation of valine, leucine or lysine. Metal-catalyzed oxidation of histidine and formation of 2- and 8-oxohistidine also can cause oxidative damage to proteins. Still further, oxidative damage also can occur by tyrosine dimerization with resultant protein aggregation. Oxidative damage to proteins can occur by the formation of carbonyls, the formation of adipic semi-aldehyde from lysine, the formation of L-DOPA from tyrosine, the formation of alkyl- (chloro- or bromo-) tyrosine, the formation of nitrotyrosine, the formation of para-, meta- and ortho-tyrosine from phenylalanine, and the formation of neoepitopes on oxidized proteins. Oxidative changes of proteins can give rise to new formations that are recognized as foreign by the immune system and elicit an immune response.

Oxidative damage to lipids can occur by formation of aldehydes (e.g., malondialdehyde and 4-HNE), pentane and ethane, 2,3 trans-conjugated dienes, isoprostane, cholesteroxides, lipofuscin, and isolevuglandin adducts, which can cause protein-DNA and protein-protein cross-linking.

Oxidative damage to nucleic acids can occur by the formation of 8-nitroguanine, 8-chloroadenine, adenine N¹-oxide, and tandem base products selected from the group consisting of thymine-guanine([5-methyl]-8), guanine-thymine(8-[5-methyl]), 6-hydroxythymine-guanine(5-8), guanine-6-hydroxythymine(8-5), adenine-thymine(8-[5-methyl]), thymine-adenine([5-methyl]-8), cytosine-guanine(5-8), and dihydrouracil-guanine(5-8). Oxidative damage to nucleic acids also can occur by the oxidation of: thymines (e.g., at the 5,6-double bond or at the 5-methyl group); cytosines at the 5,6-double bond; 5-methylcytosines; guanines to 8-oxoguanine; guanines to 2,6-diamino-4-hydroxy-formamidopyrimidine (fapyG); adenines to 4,6-diamino-formamidopyrimidine (fapyA); adenines to 8-hydroxyadenine; adenines to 2-hydroxyadenine; and, adenines to adenine N⁶-hydroxylamine. The resultant DNA structural and functional alterations include, but are not limited to, point mutations, replicative blocks, deletions, microsatellite instability/loss of heterozygosity, and epigenetic effects.

Biomolecular damage caused by oxidative stress often leads to the induction and propagation of oxidative stress-associated conditions, including, but not limited to, diseases of the blood, brain/nervous system, breast (e.g., invasive ductal carcinoma and cancer), cardiovascular system (e.g., coronary heart disease), colon (e.g., colorectal cancer), kidney (e.g., renal cell carcinoma and reperfusion injury), liver, respiratory system, skin, and stomach (e.g., H. pylori infection and cancer). Additionally, oxidative stress-associated diseases include diabetes mellitus (both insulin-dependent diabetes mellitus (IDDM) and non-IDDM), Down's Syndrome, exposure toxicity, gynecological diseases, gastrointestinal system (e.g., inflammatory bowel disease), metabolic syndrome, pancreatitis, preeclampsia, prostate cancer, rheumatoid arthritis, systemic lupus erythematosus (SLE), and viral diseases (e.g., HIV). Blood diseases include acute lymphoblastic leukemia and Fanconi's anemia. Brain/nervous system disease include Alzheimer's disease, amytrophic lateral sclerosis, cerebral amyloid angiopathy, Charcot Marie Tooth, dementia with Lewy bodies, Friedreich ataxia multiple sclerosis, and Parkinson's disease. Cardiovascular diseases include atherosclerosis, hypertension, thrombosis, and heart disease, such as coronary heart disease. Liver diseases caused by oxidative stress include, but are not limited to, chronic hepatitis, hepatitis C, hepatoblastoma, alcoholic liver disease, primary billiary cirrhosis, and heptacellular carcinoma. Respiratory system diseases caused by oxidative stress include, but are not limited to, acute respiratory distress syndrome, asthma, chronic obstructive pulmonary dysfunction (COPD), cystic fibrosis, obstructive sleep apnea, squamous cell carcinoma, and, small cell carcinoma. Skin diseases caused by oxidative stress include, but are not limited to atopic dermatitis, skin neoplasma, skin wrinkling, pre-cancerous skin changes, viteligo, and psoriasis. Other oxidative stress-associated conditions include, but are not limited to, cancer generally and aging. More detailed descriptions of the foregoing conditions can be found in, for example, Thomas E. Andreoli, M.D. (Editor), “Cecil Essentials of Medicine,” 3^{rd} Ed. (Harcourt Brace & Company, Philadelphia, Pa., 1993).

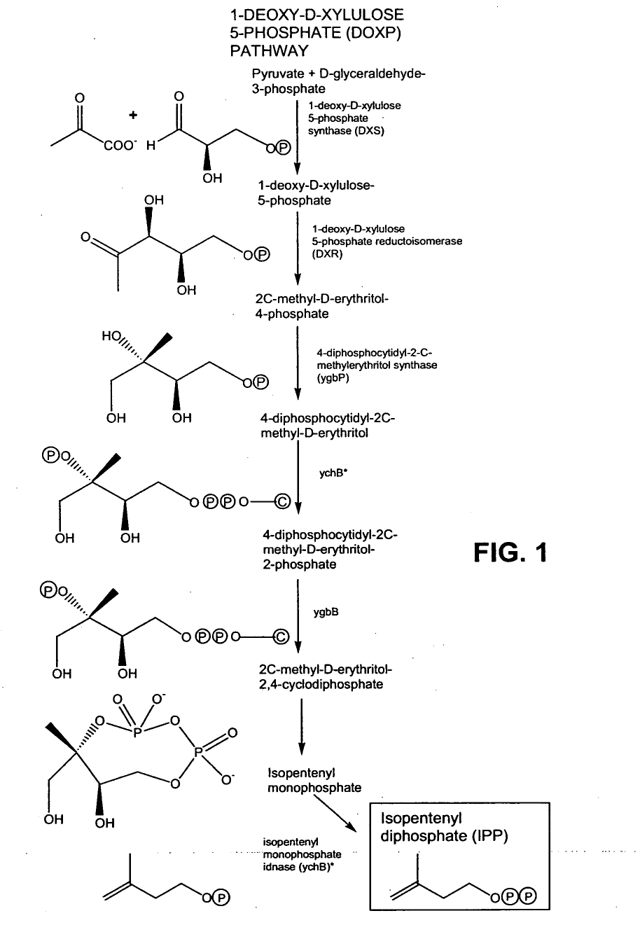

Generally, isopentenyl diphosphate is a five-carbon molecule that is a building unit of all isoprenoids, one example of which is a carotenoid. The diphosphate moiety of the molecule is capable of triggering antioxidative signaling, whereas the monophosphate moiety of an isopentenyl monophosphate molecule is unable to similarly trigger antioxidative signaling. Heretofore, isopentenyl diphosphate has not been identified as having antioxidant characteristics. Isopentenyl diphosphate can be made by a variety of biosynthetic pathways depending upon the type of organism. In some eubacteria, plants, and algae, isopentenyl diphosphate is produced by a 1-deoxy-D-xylose 5-phosphate pathway, as schematically shown in FIG. 1 and referred to herein as the DOXP pathway. As shown in FIG. 1, pyruvate and D-glyceraldehyde-3-phosphate react in the presence of 1-deoxy-D-xylulose-5-phosphate synthase (DXS) to yield 1-deoxy-D-xylulose-5-phosphate. The formed 1-deoxy-D-xylulose-5-phosphate is next reduced in the presence of 1-deoxy-D-xylulose-5-phosphate reductoisomerase (DXR) to 2C-methyl-D-erythritol-4-phosphate. This phosphate, in turn, is converted to a diphosphate in the presence of 4-diphosphocytidyl-2-C-metheylerythritol synthase (ygbp) to yield 4-diphosphocytidyl-2C-metheyl-D-erythritol. The diphosphate is next converted to a cyclodiphosphate compound in the presence of ygbB to yield 2C-methyl-D-erythritol-2,4-cyclodiphosphate. The formed, cyclic diphosphate ring is next broken to yield two moles of isopentenyl monophosphate, each of which is reacted with isopentenyl monophosphate kinase (ychB) to yield two moles of isopentenyl diphposphate.

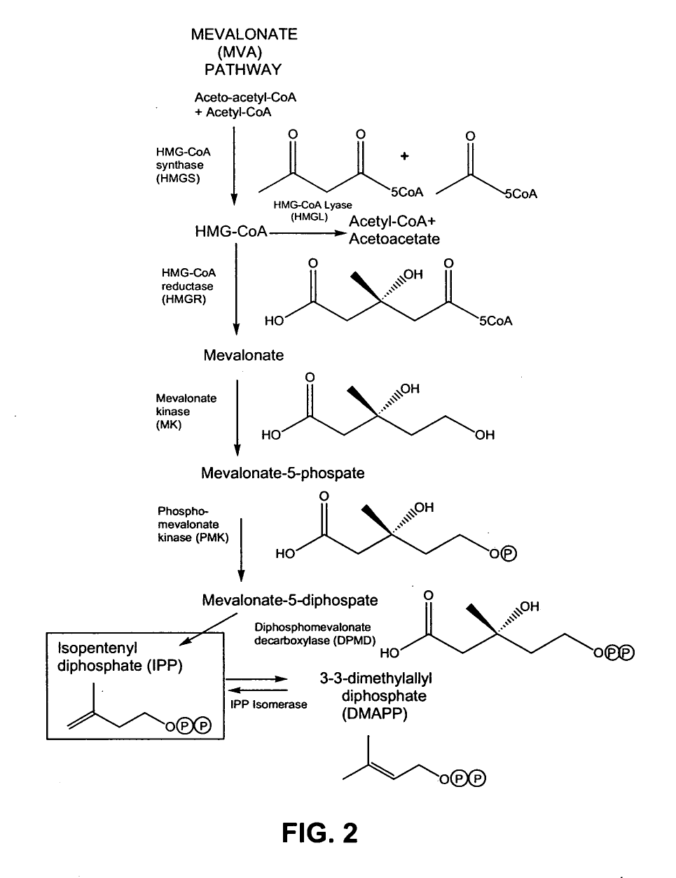

Fungi and mammals produce isopentenyl diphosphate via a mevalonate pathway, as schematically shown in FIG. 2 and referred to herein as the MVA pathway. As shown in FIG. 2, aceto-acetyl-CoA and acetyl-CoA are reacted in the presence of HMG-CoA synthase (HMGS) and HMG-CoA lyase (HMGL) to yield acetyl-CoA and acetoacetate. In the presence of HMG-CoA reductase (HMGR), the acetoacetate is converted to mevalonate. The formed mevalonate is converted to a phosphate in the presence of mevalonate kinase (MK) to a yield a mevalonate-5-phosphate. The formed phosphate is converted to a diphosphate in the presence of phosphor-mevalonate kinase (PMK) to yield mevalonate-5-diphosphate. This diphosphate can be converted in the presence of diphosphomevalonate decarboxylase (DPMD) to 3,3-dimethylallyldiphosphate (DMAPP), which can be further isomerized to yield isopentenyl diphosphate.

The methods of the invention are preferably carried out using an isopentenyl diphosphate as described above with one or more pharmaceutically acceptable carriers. The phrase “pharmaceutically acceptable” refers to molecular entities and compositions that do not produce adverse, allergic, or other untoward reactions when administered using routes well-known in the art, as described below.

The phrases “pharmaceutically acceptable salts” or “a pharmaceutically acceptable salt thereof” refer to salts prepared from pharmaceutically acceptable acids or bases, including organic and inorganic acids and bases. The active compound (i.e., isopentenyl diphosphate) used in the present invention can be used in the form of salts prepared from pharmaceutically acceptable acids. Suitable pharmaceutically acceptable acids include, but are not limited to, acetic, benzenesulfonic (besylate), benzoic, p-bromophenylsulfonic, camphorsulfonic, carbonic, citric, ethanesulfonic, fumaric, gluconic, glutamic, hydrobromic, hydrochloric, hydroiodic, isethionic, lactic, maleic, malic, mandelic, methanesulfonic (mesylate), mucic, nitric, oxalic, pamoic, pantothenic, phosphoric, succinic, sulfuric, tartaric, p-toluenesulfonic, and the like. Examples of such pharmaceutically acceptable salts of IPP, thus, include, but are not limited to, acetate, benzoate, p-hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like.

The phrases “side effects,” “adverse effects,” and “adverse side effects” in relation to IPP (and salts thereof) include, but are not limited to, dizziness, insomnia, lightheadedness, changes in blood pressure, gastrointestinal disturbances, sexual dysfunction, extrapyramidal side effects, certain anticholinergic-like effects (e.g., tachycardia, blurred vision), and undesired side effects associated with drug-drug interactions.

As used herein, the terms “treat,” “treatment,” and “treating,” refer to: (a) preventing a disease, disorder, or condition from occurring in a mammal (preferably a human being), which may be predisposed to the disease, disorder or condition but has not yet been diagnosed as having it; (b) inhibiting the disease, disorder, or condition, i.e., arresting its development; and (c) relieving the disease, disorder, or condition, i.e., causing regression of the disease, disorder or condition. In other words, the terms “treat,” “treatment,” and “treating,” extend to prophylaxis (e.g., “prevent,” “prevention,” and “preventing”) as well as treatment of established conditions. Accordingly, use of the terms “prevent,” “prevention,” and “preventing,” would be an administration of the pharmaceutical composition to a host (preferably a mammal, and highly preferably a human being) who has, in the past, suffered from the aforementioned diseases, disorders, or conditions, but is not suffering from the same at the moment of the composition's administration. For the sake of simplicity, the term “conditions” as used hereinafter encompasses diseases, disorders, and conditions.

In the various embodiments described herein, the method comprises the step of administering, and preferably orally administering, a therapeutically effective amount of isopentenyl diphosphate or a pharmaceutically acceptable salt thereof to provide a total dose of about 0.1 to about 1000 mg/day of IPP to a patient in need thereof.

In general, the preferred route of administering the inventive composition is oral, with a once- or twice-a-day administration. The dosage regimen and amount for treating patients with isopentenyl diphosphate or a pharmaceutically acceptable salt thereof is selected in accordance with a variety of factors including, for example, the type, age, weight, sex, and medical condition of the patient, the severity of the condition, the route of administration, and the particular form of IPP employed, either acid or base forms thereof. An ordinarily skilled physician can readily determine and prescribe an effective (i.e., therapeutic) amount of the compound to treat, prevent, or otherwise arrest the progress of the condition. In so proceeding, the physician could employ relatively low dosages at first, subsequently increasing the dose until a maximum response is obtained.

Pharmaceutical compositions suitable for oral administration can be of any convenient form, such as aerosol sprays, capsules, dragees, gels, liquids, pills, sachets, slurries, suspensions, syrups, and tablets, each containing a predetermined amount of the active agent either as a powder or granules, or as a solution or a suspension in an aqueous liquid, a non-aqueous liquid, an oil-in-water emulsion, or a water-in-oil liquid emulsion. Such compositions can be prepared by any method that includes the step of bringing the active agent either into intimate association with a carrier, which constitutes one or more necessary or desirable ingredients. Generally, the compositions are prepared by uniformly and intimately admixing the active agent with liquid carriers or finely divided solid carriers or both, and then, if necessary, shaping the product into a desired form.

A tablet preferably is prepared by standard pharmaceutical manufacturing techniques as described in Remington's Pharmaceutical Sciences, 18th Ed. (Mack Publishing Co., Easton, Pa., 1990). Such techniques include, for example, wet granulation followed by drying, milling, and compression into tablets with or without film coating; dry granulation followed by milling, compression into tablets with or without film coating; dry blending followed by compression into tablets, with or without film coating; molded tablets; wet granulation, dried and filled into gelatin capsules; dry blend filled into gelatin capsules; or suspension and solution filled into gelatin capsules. Compressed tablets can be prepared, for example, by compressing the active agent in a suitable machine into a free-flowing form, such as a powder or granules. Thereafter, the compressed, free-flowing form optionally can be mixed with binders, diluents, lubricants, disintegrating agents, effervescing agents, dyestuffs, sweeteners, wetting agents, and non-toxic and pharmacologically-inactive substances typically present in pharmaceutical compositions. Additional pharmaceutical excipients that can be used and are generally recognized as safe, include, for example, lactose, microcrystalline cellulose, starch, calcium carbonate, magnesium stearate, stearic acid, talc, and colloidal silicon dioxide. Molded tablets can be made by molding a mixture of the powdered compound moistened with an inert liquid diluent in a suitable machine. Generally, the solid dosage forms have identifying marks which are de-bossed or imprinted on the surface.

Suitable binders for use in the pharmaceutical preparation include, for example, starches, gelatin, methylcellulose, gum Arabic, tragacanth, and polyvinylpyrrolidone. Suitable diluents for use in the pharmaceutical preparation include, for example, lactose, dextrose, sucrose, mannitol, sorbitol, and cellulose. Suitable lubricants for use in the pharmaceutical preparation include, for example, silica, talc, stearic acid, magnesium or calcium stearate, and or polyethylene glycols. Suitable disintegrating agents for use in the pharmaceutical preparation include, for example, starches, alginic acid, and alginates. Suitable wetting agents for use in the pharmaceutical preparation include, for example, lecithin, polysorbates, and laurylsulfates. Generally, any effervescing agents, dyestuffs, and/or sweeteners known by those of ordinary skill in the art can be used in the preparation of a pharmaceutical composition.

When administered in tablet form, the composition can additionally contain a solid carrier, such as a gelatin or an adjuvant. The tablet contains about 5% to about 95% of an active agent of the present invention, and preferably from about 25% to about 90% of an active agent of the present invention. When administered in liquid form, the composition contains about 0.5% to about 90% by weight of active agents, and preferably about 1% to about 50% of an active agents.

When administered in liquid form, a liquid carrier, such as water, petroleum, or oils of animal or plant origin, can be added. The liquid form of the composition can further contain physiological saline solution, dextrose or other saccharide solutions, or glycols.

Aqueous suspensions may contain the active compound in admixture with excipients suitable for the manufacture of aqueous suspensions. Such excipients include, but are not limited to, suspending agents (e.g., sodium carboxymethylcellulose, methylcellulose, hydroxypropylmethylcellulose, sodium alginate, polyvinylpyrrolidone, gum tragacanth and gum acacia), dispersing or wetting agents, such as a naturally-occurring phosphatide (e.g., lecithin), condensation products of an alkylene oxide with fatty acids (e.g., polyoxyethylene stearate), condensation products of ethylene oxide with long chain aliphatic alcohols (e.g., heptadecaethyleneoxycetanol), condensation products of ethylene oxide with partial esters derived from fatty acids and a hexitol (e.g., polyoxyethylene sorbitol monooleate), and condensation products of ethylene oxide with partial esters derived from fatty acids and hexitol anhydrides (e.g., polyethylene sorbitan monooleate). The aqueous suspensions may also contain one or more preservatives (e.g., ethyl, or n-propyl, p-hydroxybenzoate), one or more coloring agents, one or more flavoring agents, and one or more sweetening agents (e.g., sucrose or saccharin).

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, also may be present.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

The pharmaceutical compositions of the invention also may be in the form of oil-in-water emulsions. The oily phase may be a vegetable oil, for example, olive oil, arachis oil, sesame oil or coconut oil, or a mineral oil, for example, liquid paraffin, or mixtures of these. Suitable emulsifying agents may be naturally-occurring gums, for example, gum acacia or gum tragacanth, naturally-occurring phosphatides, for example, soy bean, lecithin, and esters or partial esters derived from fatty acids and hexitol anhydrides, for example, sorbitan monooleate, and condensation products of the said partial esters with ethylene oxide, for example polyoxyethylene sorbitan monooleate. The emulsions also may contain sweetening and flavoring agents.

Syrups and elixirs may be formulated with sweetening agents, for example glycerol, propylene glycol, sorbitol or sucrose. Such formulations also may contain a demulcent, a preservative and flavoring and coloring agents.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation also may be a sterile injectable solution or suspension in a non-toxic parenterally-acceptable diluent or solvent, such as, for example, a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

The terms “day” and “daily” refer to the administration of the product one or more times, preferably one to three times, still more preferably one time, per about 24-hour period. The phrase “about 24-hour period” refers to a time span of about 20 to about 28 hours. Desirably, a unit dose of the composition (e.g., tablet, sachet, or capsule) contains from about 0.1 milligrams (mg) to about 1000 mg of isopentenyl diphosphate. More preferably, each unit dose contains about 50 mg to about 250 mg of the active ingredient, isopentenyl diphosphate. Even more preferably, however, each unit dose contains from about 50 mg to about 250 mg of the active ingredient. This dosage form permits a full daily dosage of about 50 mg to about 250 mg to be administered in one or more unit doses. This will allow for unit dosage tablets, for example, containing about 10, 20, 30, 40, 50, 60, 70, 80, 90, 100, 110, 120, 130, 140, 150, 160, 170, 180, 190, 200, 210, 220, 230, 240, or about 250 mg of isopentenyl diphosphate.

The isopentenyl diphosphate can be administered by any suitable route, for example by oral, buccal, inhalation, sublingual, rectal, vaginal, intracisternal through lumbar puncture, transurethral, nasal, percutaneous, i.e., transdermal, or parenteral (including intravenous, intramuscular, subcutaneous, intracisternal, and intracoronary) administration. Administration by intravenous, intradermal, intramuscular, intramammary, intraperitoneal, intrathecal, retrobulbar, intrapulmonary injection and/or surgical implantation at a particular site is contemplated as well. The term “oral dosage form” is used in a general sense to reference pharmaceutical products administered orally. Oral dosage forms are recognized by those skilled in the art to include such forms as aerosol sprays, capsules, dragees, gels, liquids, pills, sachets, slurries, suspensions, syrups, and tablets. Parenteral administration can be accomplished using a needle and syringe, or using a high pressure technique, like POWDERJECT™.

Pharmaceutical compositions containing insopentenyl diphposphate or a pharmaceutically acceptable salt thereof can be formulated into a cream, cleanser (water-less and rinse-off type cleansers), solution, spray, suspension, gel, lotion, mousse, or ointment using a pharmaceutically acceptable carrier and, optionally, additional ingredients. The “International Cosmetic Ingredient Dictionary and Handbook,” 10^{th} Ed. (CTFA, 2004), which is hereby incorporated herein by reference, describes a wide variety of cosmetic and pharmaceutical ingredients commonly used in the skin care industry that also are suitable for use in the topical pharmaceutical compositions described herein. Suitable ingredients include, but are not limited to, absorbents, abrasives, anti-acne agents, anticaking agents, antifoaming agents, antimicrobial agents, antioxidants other than isopentenyl diphosphate, binders, biological additives, buffering agents, bulking agents, chelating agents, chemical additives, colorants, cosmetic astringents, cosmetic biocides, denaturants, drug astringents, emulsifiers, external analgesics, film formers, foam boosters, fragrance comoponents, humectants, hydrotropes, opacifying agents, pH adjusters, plasticers, preservatives, propellants, reducing agents, sequestrants, skin bleaching agents, skin-conditioning agents (e.g., emollient, humectants, miscellaneous, and occlusive), skin protectants, skin sensates, solvents, solubilizing agents, suspending agents, sunscreen agents, ultraviolet light absorbers, and viscosity increasing agents. Such compositions are amenable to topical administration directly to the skin. As used herein, the term “rinse-off” generally refers to a form of the composition such that it can be used in a cleansing ultimately be rinsed or washed from the skin with water to complete the cleansing process. As used herein, the term “water-less,” generally refers to a form of the composition such that it can be used in a cleansing process without water and whereby the composition can be removed by wiping with an article, such as a cotton ball, a cotton pad, a tissue, a towel, or the like.

In further embodiments, the topical composition can be useful for personal cleansing, especially for cleansing of the face and neck areas. Typically, a suitable or effective amount of the cleansing composition is applied to the area to be cleansed. Alternatively, a suitable amount of the cleansing composition can be applied via intermediate application to a washcloth, sponge, pad, cotton ball or other application device. If desired, the area to be cleansed can be premoistened with water. Generally, an effective amount of composition to be used will depend upon the needs and usage habits of the individual and the amount of isopentenyl diphosphate or a pharmaceutically acceptable salt thereof present in the composition.

The pharmaceutical compositions include those wherein isopentenyl diphosphate is administered in an effective amount to achieve its intended purpose. More specifically, a “therapeutically effective amount” means an amount effective to prevent development of, to eliminate, to treat or to otherwise alleviate oxidative stress-associated diseases. Determination of a therapeutically effective amount is well within the capability of those skilled in the art, especially in light of the detailed disclosure provided herein.

A “therapeutically effective dose” refers to the amount of the active agent(s) that results in achieving the desired effect. Toxicity and therapeutic efficacy of such active agents can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The obtained data can be used in formulating a dose range for use in humans. The dosage of the active agent preferably lies within a range of circulating concentrations that include the ED₅₀ with little or no toxicity. The dosage can vary within this range depending upon the dosage form employed, and the route of administration utilized.

The exact formulation, route of administration, and dosage can be determined by an individual physician in view of the patient's condition. Dosage amounts and intervals can be adjusted individually to provide levels of active agents that are sufficient to maintain therapeutic or prophylactic effects.

Specifically, for administration to a human in the curative or prophylactic treatment of an oxidative stress-associated disease, oral dosages of isopentenyl diphosphate generally are about 100 mg/day to about 1000 mg/day, more preferably about 200 mg/day to about 1000 mg/day, and most preferably an about 250 mg/day to about 1000 mg/day for an average adult patient (70 kg), typically divided into two to three doses per day. In practice, and as previously-noted herein, a physician can determine the actual dosing regimen that is most suitable for an individual patient, and the dosage varies with the age, weight, and response of the particular patient. The above dosages are exemplary of the average case, but there can be individual instances in which higher or lower dosages are merited, and such are within the scope of this invention.

When a therapeutically effective amount of isopentenyl diphosphate is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to the active agent, an isotonic vehicle.

Isopentenyl diphosphate can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents, such as suspending, stabilizing, and/or dispersing agents.

Pharmaceutical compositions for parenteral administration include aqueous solutions of isopentenyl diphosphate in water-soluble form. Additionally, suspensions of isopentenyl diphosphate can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Isopentenyl diphosphate also can be formulated in rectal compositions, such as suppositories or retention enemas, e.g., containing conventional suppository bases. In addition to the formulations described previously, isopentenyl diphosphate also can be formulated as a depot preparation. Such long-acting formulations can be administered by implantation (for example, subcutaneously or intramuscularly) or by intramuscular injection. Thus, for example, isopentenyl diphosphate can be formulated with suitable polymeric or hydrophobic materials (for example, as an emulsion in an acceptable oil) or ion exchange resins, or as sparingly soluble derivatives, for example, as a sparingly soluble salt.

In particular, isopentenyl diphosphate can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. An active agent also can be injected parenterally, for example, intravenously, intramuscularly, subcutaneously, intrathecally, intracisternally, or intracoronarily. For parenteral administration, the active agent is best used in the form of a sterile aqueous solution which can contain other substances, for example, salts, or monosaccharides, such as mannitol or glucose, to make the solution isotonic with blood.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (1996) J. Pharm. Sci. 85:1282-1285, and Oliyai et al. (1993) Ann. Rev. Pharmacol. Toxicol. 32:521-544.

As noted above, one embodiment of the invention is directed to an article of manufacture that includes a packaged pharmaceutical composition containing isopentenyl diphosphate or a pharmaceutically acceptable salt thereof. The article also includes an insert providing instructions for administration of the composition to treat an oxidative stress-associated disease in a host, and a container for the packaged pharmaceutical composition and the insert.

The term “insert” when referring to package insert, means information accompanying the composition (product) that provides a description of how to administer the product, along with the safety and efficacy data required to allow the physician, pharmacist, and patient to make an informed decision regarding the use of the product. The package insert generally is regarded as the “label” for a pharmaceutical product. The package insert generally is regarded as the label of the pharmaceutical product. The package insert incorporated into the present article of manufacture indicates that IPP is useful in the treatment of the aforementioned conditions wherein DNA damage protection or other oxidative species regulation is desired. The package insert also provides instructions to administer one or more about 50 mg to about 1000 mg unit dosage forms, chronically, and preferably daily, for at least three days, up to a maximum total dose of 1000 mg per day. The dose administered typically is about 50 mg/day to about 1000 mg/day, more preferably about 200 mg/day to about 1000 mg/day, and most preferably an about 250 mg/day to about 1000 mg/day.

The container used in the present article of manufacture is conventional in the pharmaceutical arts. Generally, the container is a blister pack, foil packet, glass or plastic bottle and accompanying cap or closure, or other such article suitable for use by the patient or pharmacist. Preferably, the container is sized to accommodate 1-1000 solid dosage forms, preferably 1 to 500 solid dosage forms, and most preferably, 5 to 30 solid dosage forms.

### EXAMPLES

Described below are procedures for demonstrating the antioxidative potency and mechanism of action of isopentenyl diphosphate in an in-vitro tissue-culture model system and the results thereof.

Reagents

The following reagents were used: 2′,7′-dichlorofluorescin diacetate (“DCFH-DA”) commercially-available from Molecular Probes of Eugene, Oreg.; xanthine amine congener (“XAC”), 2-[p-2-(carbonyl-ethyl)-phenyethylamino]-5′-N-ethylcarboxaminoadenosine (“CGS21680”), 5′-(N-ethylcarboxyamido)-adenosine (“NECA”); [(3)H]1,3-dipropyl-8-cyclopentylxanthine (“DPCPX”), 8-(3-chlorostyryl) caffeine (“CSC”), and N6-(3-iodobenzyl)adenosine-5′-N-methyluronamide (“IB-MECA”) all of which are commercially-available from RBI of Natick, Mass.; N-[2-(p-bromocynnamylamino)ethyl]-5-isoquinolinesulfonamide (“H-89”), lactacystin, and lovastatin all of which are commercially-available from Calbiochem of San Diego, Calif.; isopentenyl monophosphate (“IMP”), farnesyl diphosphate (“FPP”), and geranylgeranyl diphosphate (“GGPP”) all of which are commercially-available from Larodan of Malmo, Sweden; and, Comet LMAgarose, Lysis Solution, Trevigen CometSlide™, and SYBR Green Concentrate all of which are commercially-available from Trevigen, Inc., of Gaithersburg, Md. All other reagents, including 2-chloroadenosine (also abbreviated herein as “2CA”) and isopentenyl diphosphate, are commercially-available from Sigma-Aldrich Co. of St. Louis, Mo.

Cell Lines

Human fibroblastoid M1 cells were grown in Dulbecco's modified Eagle's medium (DMEM) containing 10% fetal bovine serum (FBS), penicillin/streptomycin, and 10 mM HEPES buffer solution. Human embryonic kidney 293 (HEK293) cells, expressing cDNA corresponding to A1AR, A2aAR, or A2bAR were grown as a confluent monolayer culture in DMEM/F12 medium supplemented with 10% FBS, penicillin/streptomycin, and 250 micrograms per milliliter (μg/ml) G418. See J. Linden et al. (1999) Mol. Pharmacol. 56:705-513.

Adenosine Receptors

Adenosine is a ubiquitous nucleoside that produces a wide variety of physiological effects through the activation of cell surface adenosine receptors (abbreviated herein as “AR”). Adenosine generally is released in response to ischemic and inflammatory stresses and acts as a paracrine cytoprotective agent. In cardiovascular and renal systems, adenosine reduces cellular injury during ischemia-reperfusion. Adenosine receptors are members of the G-protein-coupled receptor family, and four subtypes are commonly recognized among this group: A1AR, A2aAR, A2bAR, and A3AR. See generally, M. E. Olah et al. (2000) Pharmacol. Ther. 85:55-75.

The adenosine A1 receptor, A1AR, is highly expressed in brain tissue (especially cerebellum, hippocampus, thalamus, and cortex) and spinal cord tissue. and in part, modulates neurotransmitter release. It has been reported that A1AR regulates tumor necrosis factor-α (TNFα) and interleukin-6 (IL-6) expression and exhibits diminished function in patients with multiple sclerosis. See J. B. Johnston et al. (2001) Ann. Neurol. 49:650-658. Other tissues also express A1AR, including, for example, kidney and testis.

The adenosine A2 receptor, A2aAR, is a glycoprotein that activates adenylyl cyclase through Gs interactions. Stimulation of this receptor and the resultant accumulation of cAMP inhibits platelet aggregation and, in certain vascular beds, is believed to be associated with vasodilation and a decrease in blood pressure. This adenosine receptor has been reported to interact with D2 dopamine receptor sites in the brain where they co-express. A2aAR also is expressed in other tissues, such as, for example, heart, kidney, and lung tissue.

The adenosine A2 receptor, A2bAR, is a glycoprotein that activates adenylyl cyclase through Gs interactions. It is expressed in the caecum, colon, urinary bladder, brain, spinal cord, and lung. The physiologic function of this receptor is less well understood, compared with A2aAR. Stimulation of this receptor and the resultant accumulation of cAMP activate mast cell degranulation.

The adenosine A3 receptor, A3AR, also has a widespread distribution. It is expressed in sperm, mast cells, lung, kidney, brain, and heart. Stimulation of this receptor leads to activation of Gi with resultant inhibition of adenylyl cyclase. Activation of this receptor has been shown to activate eosinophil migration.

Measurement of ROS

The presence of reactive oxidative species was measured using commercially-available equipment in accordance with the manufacturer's instructions. Specifically, sub-confluent cells were detached from flasks using 0.05% trypsin and 5×10³ human fibroblastoid M1 cells per well were seeded into 96-well plates (Falcon 35-3072). After an overnight culture, the cells were twice washed with DMEM-phenol red-free medium. The cells were subsequently treated with various concentrations of signal-modulating agents in the presence of 10 micromolar (μM) DCFH-DA in 100 microliters (μl) DMEM-phenol red-free medium at 37° C. for 30 minutes. This medium was replaced by a 100 μl DMEM-phenol red-free medium containing DCFH-DA and either 50 mU/ml GO, or 100 μM H₂O₂, and the cultures were subsequently incubated at 37° C. for 30 minutes. After thirty minutes incubation, the plates were read by CytoFluor™ 2300 (Millipore) at an excitation wavelength (Eₓ) of 480 nm and emission wavelength (Eₘ) of 520 nm, or by Fusion-Alpha HT system (PerkinElmer Life Sciences, Boston, Mass.) at Eₓ 485 nm and Eₘ 530 nm.

Quantification of DNA Damage by the Comet Assay

A single-cell gel electrophoresis (SCGE) assay, known as the “Comer” assay was performed to detect/quantify DNA damage by identifying DNA-strand breakage in individual cells, for example. The assay was performed using a kit commercially-available from Trevigen, Inc., of Gaithersburg, Md., as “CometAssay™.” See P. J. McCarthy et al. (1997) Mutagenesis 12:209-214; A. R. Collins et al. (2001) Biochem. Soc. Trans. 29:337-341. Sub-confluent cells were washed with phosphate buffered saline (PBS) and detached from the tissue culture flasks using a 0.05% trypsin solution. After centrifugation, the cells were washed and re-suspended in serum-free DMEM medium. The cells were divided into Eppendorf tubes (1.5 ml), and centrifuged at 2500 rotations per minutes (rpm) for ten minutes. Signal-modulating agents were added and samples were kept in a 37° C. incubator for thirty minutes. In some experiments, the cells were pre-incubated with various inhibitors for the indicated times at 37° C. At the end of the incubation period with signal-modulating chemicals, the cells were washed with PBS and treated with 100 μM H₂O₂ in 0.5 ml PBS on ice for twenty minutes. Following the hydrogen peroxide treatment, the cells were immediately microfuged for fifteen seconds. The cells were twice washed with cold PBS, and re-suspended at 1.0×10⁵ cells/ml in ice-cold PBS. Five microliter cell suspension were each mixed with 50 μl molten LMAgarose and, thereafter, the mixtures were placed onto slides (CometSlide™).

The slides were placed at 4° C. in the dark for ten minutes and subsequently immersed in pre-chilled Lysis Solution at 4° C. for an additional thirty minutes. Following this incubation, excess buffer was tapped off from the slides. Subsequently, the slides were placed in a 50 ml Coplin jar containing alkali buffer for thirty minutes at room temperature in the dark and then immersed in 50 ml of TBE buffer for five minutes. The slides were then placed flat onto a gel tray submerged in TBE buffer in a horizontal electrophoresis apparatus, and aligned equidistant from the electrodes. Electrophoresis was performed at one volt per centimeter (cm) for ten minutes. Following this electrophoresis, the samples were fixed in 100% methanol for five minutes and 100% ethanol for five minutes. After drying, 50 μl of diluted SYBR Green Concentrate was added onto each circle of agarose. Comet tail fluorescence intensity was quantified with a Diagnostic Instruments digitized camera, mounted on a Nikon Eclipse E400 microscope, using Scion Image software. Quantification of comet fluorescence was performed as described in P. J. McCarthy et al. (1997) Mutagenesis 12:209-214. Each data point was derived from 30 to 100 individual cells. The data are shown either in arbitrary fluorescent units, or as % DNA damage protection (i.e., the percent decrement in tail fluorescence intensity with test reagent, relative to the intensity recorded in cells treated with hydrogen peroxide only)±S.E.M.

Measurement of cAMP Levels

Cyclic AMP levels were measured using a kit that is commercially-available from Amersham Biosciences of Piscataway, N.J., under the name “EIA Kit” (Cat. No. RPN225). Specifically, human fibroblastoid M1 cells were cultured overnight in 6-well tissue culture plates, 2×10⁵ cells per well. Before stimulation, the cells were washed once with serum-free DMEM medium and incubated in 37° C. with 0.2 millimolar (mM) RO20-1724, a phosphodiesterase inhibitor, for fifteen minutes. Following this incubation period, 10 μM of either 2-chloroadenosine or isopentenyl diphosphate were added to each cell culture, and the cells cultures were thereafter maintained at 37° C. for the indicated time in the presence of phosphodiesterase inhibitor (RO20-1724). Determination of cAMP levels was performed with the EIA Kit in accordance with its manufacturer's instructions.

Statistical Analyses

The data and other results are expressed herein as mean±S.E.M. Student's unpaired t test was performed using Microsoft Excel® software and statistical significance was achieved at P<0.05 (marked in the drawing figures with an asterisk (*)). Dose-response graphs and calculation of 50% inhibitory concentration (IC₅₀) values were determined using PRISM 3.0 software.

Inhibition of Oxidative DNA Damage by Isopentenyl Diphosphate

As mentioned above, several isoprenoids have been implicated in oxidative stress regulation. Because isopentenyl diphosphate is a building block of all isoprenoids, its antioxidative effect was investigated. DCFH was used to determine whether isopentenyl diphosphate affects the oxidative status of cells. DCFH-DA diffuses across cell membranes and is hydrolyzed by intracellular esterases to liberate DCFH. In turn, DCFH is oxidized to the highly fluorescent DCF compound upon exposure to various oxidative species.

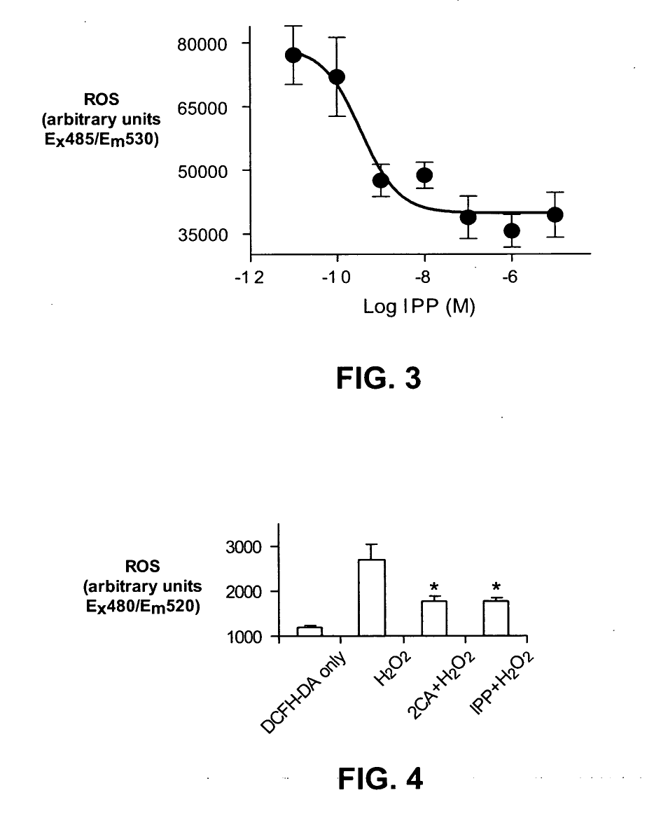

Accordingly, DCFH-DA-loaded human fibroblastoid M1 cells were incubated for thirty minutes with different concentrations of isopentenyl diphosphate before being exposed to 50 mU/ml of GO for thirty minutes. ROS levels were quantified using the Fusion-Alpha HT system, as described above. FIG. 3 graphically illustrates ROS generation (arbitrary units) as a function of the molar concentration of isopentenyl diphosphate (Log IPP (M)). As shown in FIG. 3, isopentenyl diphosphate is efficient at blocking ROS generation.

Prior studies have reported that the cAMP/PKA pathway is capable of inhibiting oxidative damage in different cell types. See generally, L. E. Hastie et al. (1997) J. Cell. Physiol. 172:373-381; L. Ochoa et al. (1997) Am. J. Respir. Crit. Care Med. 156:1247-1255; O. Inanami et al. (1999) Antioxid. Redox Signal. 1:113-121; M. Wartenberg et al. (2000) Int. J. Cancer 85:267-274; E. Franzini et al. (1996) Free Rad. Biol. Med. 21:15-23; G. W. Sullivan (2001) Br. J. Pharmacol. 132:1017-1026; M. D. Okusa et al. (2001) Kidney Int. 59:2114-2125. To verify the efficiency of that pathway, DCFH-DA-loaded human fibroblastoid M1 cells were incubated with 10 μM 2-chloroadenosine for thirty minutes followed by incubation with 100 μM H₂O₂ for twenty minutes to induce oxidative stress. Similarly, DCFH-DA-loaded human fibroblastoid M1 cells were incubated with 10 μM isopentenyl diphosphate for thirty minutes followed by incubation with 100 μM H₂O₂ for twenty minutes to induce oxidative stress. ROS levels were quantified using the CytoFluor™ 2300 system, as described above. FIG. 4 graphically illustrates the levels of ROS generation (arbitrary units) in human fibroblastoid M1 cells treated with and without 10 μM 2-chloroadenosine or 10 μM isopentenyl diphosphate. As shown in FIG. 4, the human fibroblastoid M1 cells responded to 2CA-mediated antioxidant signaling. Furthermore, the human fibroblastoid M1 cells treated with 10 μM of 2-chloroadenosine or isopentenyl diphosphate demonstrated equivalent inhibition of H₂O₂-induced ROS formation.

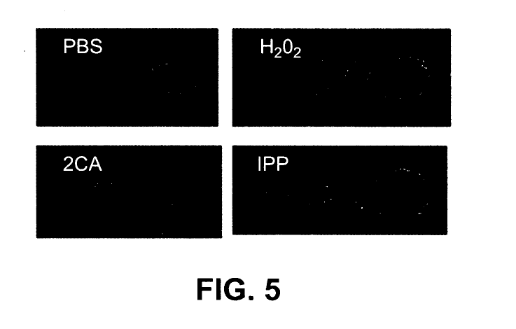

As previously noted herein, one of the harmful effects of increased ROS levels is oxidative damage to biomolecules, such as nucleic acids (e.g., DNA). The Comet assay described above was used to determine whether 2-chloroadenosine or isopentenyl diphosphate can inhibit oxidative DNA damage. FIG. 5 shows fluorescence photomicrographs of representative human fibroblastoid M1 cells subjected to the Comet assay. The cells were treated with and without 10 μM 2-chloroadenosine or 10 μM isopentenyl diphosphate followed by incubation with 100 μM H₂O₂ for twenty minutes to induce oxidative stress. For comparative purposes, control cells, which were exposed to PBS instead of H₂O₂ are shown herein as well. As shown in FIG. 5, both 2-chloroadenosine and isopentenyl diphosphate prevented H₂O₂-mediated DNA damage in human fibroblastoid M1 cells.

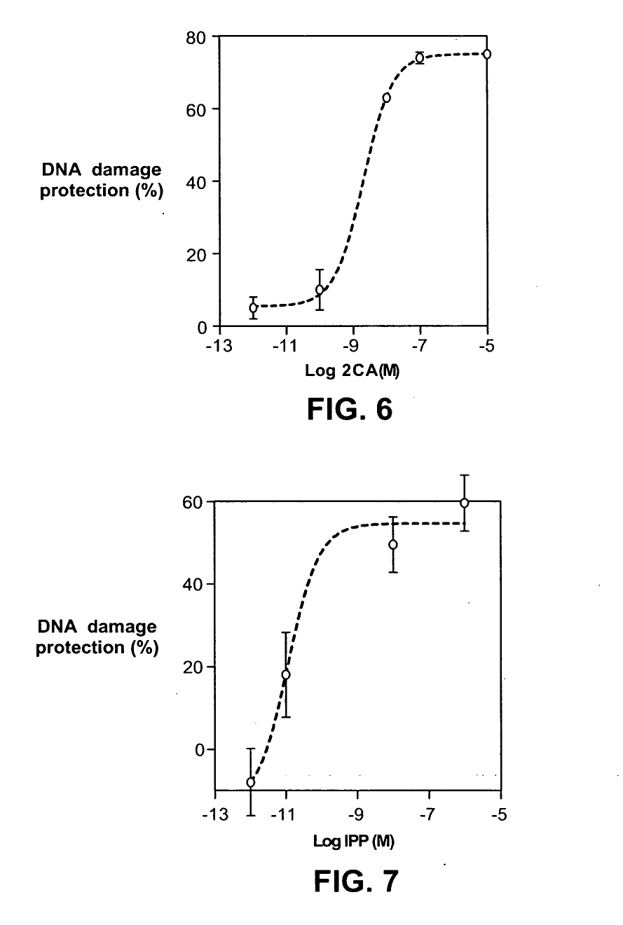

A dose response analysis of DNA damage protection also was performed to determine the efficacy of various doses of isopentenyl diphosphate versus 2-chloroadenosine. The analysis was carried out by pre-incubating human fibroblastoid M1 cells for thirty minutes with different concentrations of isopentenyl diphosphate or 2-chloroadenosine prior to incubating the cells with 100 μM H₂O₂ to induce oxidative stress. Following induction of oxidative stress, DNA damage was quantified using the Comet assay, as described above. FIGS. 6 and 7 generally illustrate DNA damage protection as a function of the concentration of 2-chloroadenosine and isopentenyl diphosphate, respectively. More specifically, FIG. 6 is a graph illustrating the DNA damage protection as a percentage on the y-axis, and the logarithmic concentration of 2-chloroadenosine on the x-axis. Similarly, FIG. 7 is a graph illustrating the DNA damage protection as a percentage on the y-axis, and the logarithmic concentration of isopentenyl diphosphate on the x-axis. The dose response analysis of DNA damage protection by 2-chloroadenosine and isopentenyl diphosphate, as shown in FIGS. 6 and 7, revealed IC₅₀ values (i.e., the concentration that produces 50% inhibition) for 2-chloroadenosine and isopentenyl diphosphate of 2.04×10⁻⁹ M and 1.7×10⁻¹¹ M, respectively. Thus, in view of these findings, it can be seen that isopentenyl diphosphate is about 100 times more potent than 2-chloroadenosine.

Oxidative damage to DNA by hydrogen peroxide requires reduction by ferrous or copper ions via Fenton chemistry (e.g., H₂O₂+Fe²⁺→OH⁻+OH⁻+Fe³⁺). Removal of iron or copper ions by metal chelators has been shown to decrease DNA damage. See P. T. Doulias et al. (2003) Free Radic Biol Med. 35:719-728. The failure to recognize the role of this reaction in the past has led to mistaken identification of certain compounds as “antioxidants,” while their effect has been later shown to be due to metal chelation. See G. Fowler et al. (2003) Free Radic Biol Med. 34:77-83. It was determined, however, that the effect of isopentenyl diphposphate is not attributable to chelation of ferrous or copper ions.

Characterization of 2CA-Mediated Antioxidative Signaling

The involved transmembrane signaling was characterized to determine and understand the mechanisms of 2CA-mediated DNA damage protection. As stated above, there are four known adenosine receptors. Of the four, A1AR and A3AR are cAMP-inhibitory, Gi-protein coupled receptors, whereas A2aAR and A2bAR are coupled to Gs and activate the cAMP-mediated signaling pathway. A pharmacological approach was undertaken to characterize the pathway involved in 2CA-mediated DNA damage protection and the results are set forth Table 1, below.

TABLE 1

| Agent | IC₅₀ (pM) | Notes |
|---|---|---|
| AR Agonist | | |
| 2CA | 2040 | AR-Nonselective |
| NECA | <1000 | AR-Nonselective |
| IB-MECA | >10,000,000 | A3-Selective |
| CGS21680 | >30,000,000 | A2a-Selective |
| AR Antagonist | | |
| Enprofylline | 1,800,000 | A2b-Selective |
| XAC | 3,200,000 | AR-Nonselective |
| DPCPX | >10,000,000 | A1-Selective |
| CSC | >10,000,000 | A2a-Selective |
| Isoprenyls | | |
| IPP | 17 | |
| IMP | >10,000,000 | |
| GGPP | 660 | |
| FPP | >10,000,000 | |

The non-selective adenosine receptor agonists (2-chloroadenosine and NECA) triggered DNA damage protection; however, the A2aAR-selective agonist (CGS21680) and A3AR-selective agonist (IB-MECA) failed to transduce the signal. The non-selective adenosine receptor antagonist (XAC), as well as A2bAR-selective antagonist (enprofylline), effectively blocked the signal, while the A1AR-selective antagonist (DPCPX) and the A2aAR-selective antagonist (CSC) failed to block 2CA-mediated DNA damage protection.

It is believed that the data, when considered together, suggest that A2bAR—rather than the A2aAR, A1AR, or MAR adenosine receptors—is involved in transducing 2CA-mediated antioxidative signaling. However, it should be cautioned that the failure of A2aAR agonist (CGS21680) to trigger the signal and the failure of the A2aAR antagonist (CSC) to inhibit it in human fibroblastoid M1 cells, could be taken to mean that either 2-chloroadenosine antioxidative signaling is transduced selectively by A2bAR and not by A2aAR, or that the very low level of expression of A2aAR in human fibroblastoid M1 cells, as RT-PCR experiments suggest (data not shown), is responsible for the result.

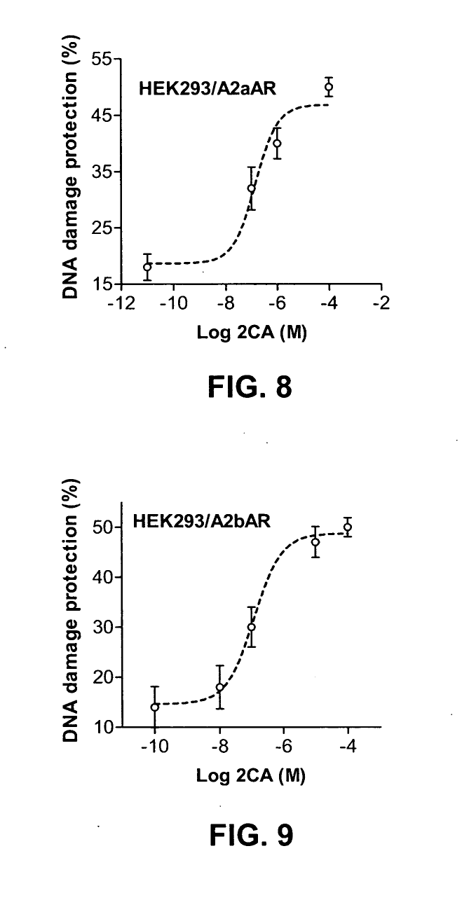

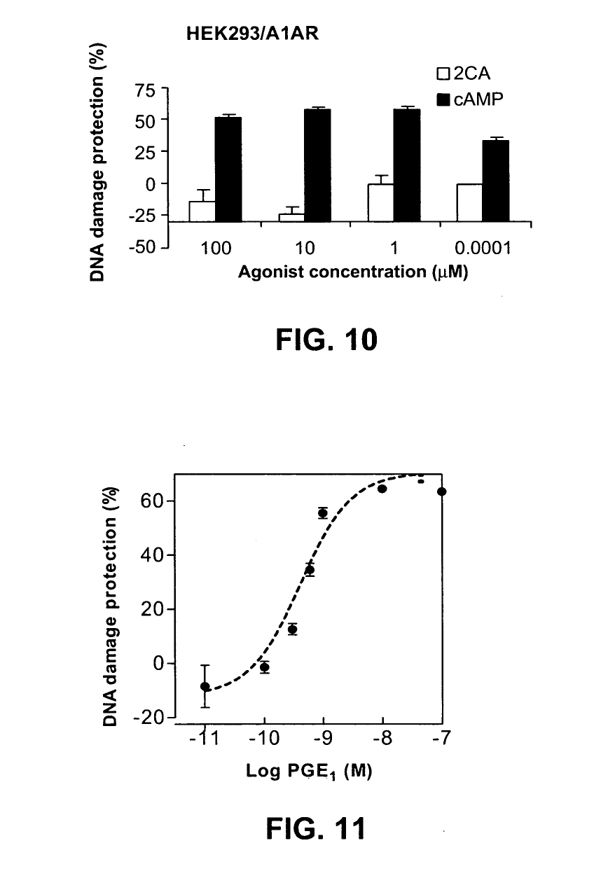

To address which of the two plausible theories may be correct, and to more directly assess the role of adenosine receptors in 2CA-mediated DNA protection, human embryonic kidney (HEK) 293 cells transfected with different adenosine receptor cDNAs were examined and the results obtained therefrom are shown in FIGS. 8-10. Specifically, HEK293 cell transfectants expressing cDNA corresponding to either A2aAR (FIG. 8), A2bAR (FIG. 9), or A1AR (FIG. 10) were incubated for thirty minutes with different concentrations of 2-chloroadenosine (as shown in FIGS. 8-10) or cAMP (as shown in FIG. 10), followed by incubation for an additional twenty minutes with 100 μM H₂O₂. At the end of incubation, DNA damage was quantified using the Comet assay, as described above. As shown in FIGS. 8-10, the A2aAR-transfected HEK293 cells and A2bAR-transfected HEK293 cells responded to 2-chloroadenosine (IC₅₀=1.4×10⁻⁷ and 1.2×10⁻⁷, respectively), while the A1AR-transfected HEK293 cells failed to transduce the antioxidative signal. The failure of A1AR-transfected HEK293 cells to respond to 2-chloroadenosine was not a result of an intracellular signaling defect, as they responded efficiently to a membrane-permeable cAMP analog, 8-bromo adenosine 3′,5′-cyclic monophosphate (8-Br-cAMP) (see the shaded bars in FIG. 10). Thus, based on the foregoing, both A2aAR and A2bAR transduce 2CA-mediated DNA damage protection signals. Furthermore, the failure of the A2aAR agonist (CGS21680) to trigger the signal and the failure of the A2aAR antagonist (CSC) to inhibit it in human fibroblastoid M1 cells appears to be due to the very low level of expression of A2aAR in those cells.

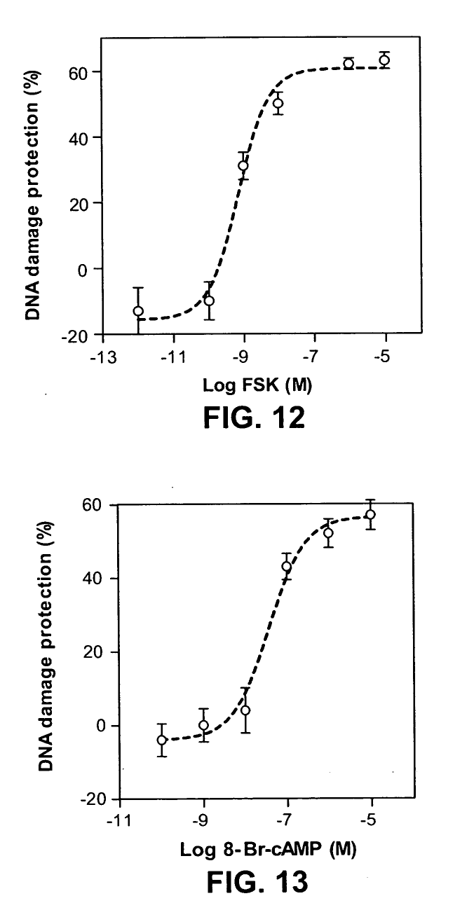

Induction of resistance to oxidative DNA damage was not unique to the adenosine-mediated pathway and could be triggered by other agents. To be sure, human fibroblastoid M1 cells were incubated for thirty minutes in either Prostaglandin E1 (PGE₁), forskolin (“FSK”), or 8-Br-cAMP at various concentrations, followed by incubation for an additional twenty minutes with 100 μM H₂O₂ to induce oxidative stress. Following induction of oxidative stress, DNA damage was quantified using the Comet assay, as described above. Shown in FIGS. 11-13 are graphs illustrating the DNA damage protection as a function of the molar concentration of PGE₁ (FIG. 11), FSK (FIG. 12), and 8-Br-cAMP (FIG. 13). Based on the obtained data, induction of resistance to oxidative DNA damage was not unique to the adenosine-mediated pathway and could be triggered by another ligand known to signal through a Gs-protein-coupled receptor, PGE₁. Additionally, intracellular activation of the cAMP-mediated pathway by forskolin (FIG. 12) or by 8-Br-cAMP (FIG. 13) produced similar protection against oxidative DNA damage.

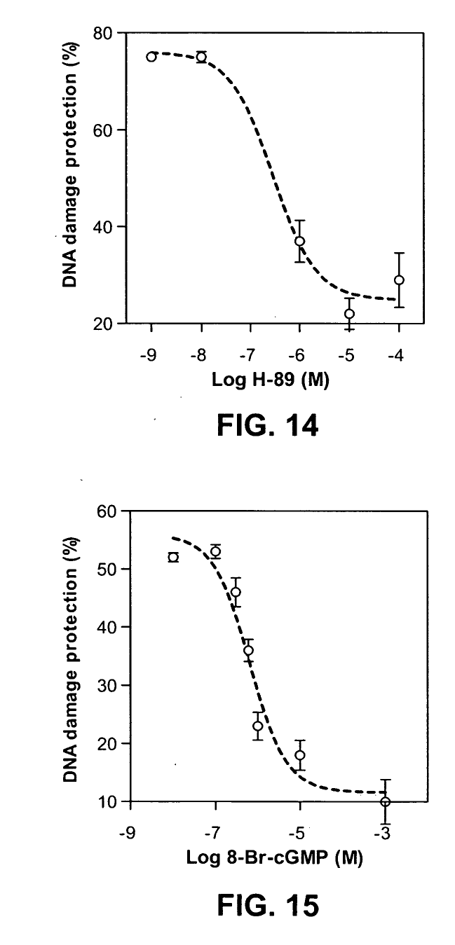

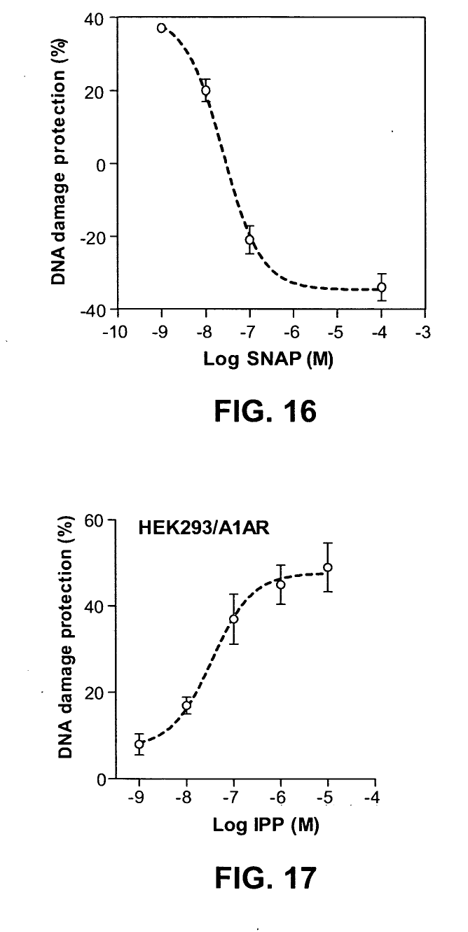

Inhibition of 2CA-mediated antioxidative signaling by cAMP pathway antagonists also was investigated. Specifically, prior to their exposure to 2-chloroadenosine, human fibroblastoid M1 cells were incubated for thirty minutes in either H-89 or 8-bromoguanosine-3′,5′-cyclic monophosphate (8-Br-cGMP) (B), or for fifteen minutes in SNAP, at different concentrations. Following incubation, the cells were exposed to 10 μM of 2-chloroadenosine for thirty minutes, followed by 100 μM H₂O₂ for twenty minutes to induce oxidative stress. Following induction of oxidative stress, DNA damage was quantified using the Comet assay, as described above. Shown in FIGS. 14-16 are graphs illustrating the DNA damage protection as function of the molar concentration of H-89 (FIG. 14), 8-Br-cGMP (FIG. 15), and SNAP (FIG. 16). Based on the obtained data, dose-dependent inhibition of 2CA-mediated antioxidative signal is attainable by inhibition of PKA by H-89, increasing nitric oxide levels by SNAP, or treating cells with a membrane permeable 8-Br-cGMP. Thus, these results suggest that an intracellular balance between the counteracting cAMP/PKA and NO-cGMP pathways may determine the extent of DNA damage protection.

Comparisons Between the IPP- and 2CA-Mediated Pathways

Unlike with 2-chloroadenosine, antioxidative signaling with isopentenyl diphosphate has been found to be independent of A2aAR or A2bAR. Specifically, IPP-mediated DNA damage protection could be found in A1AR-transfected HEK293 cells. HEK293 transfectant cells expressing A1AR were incubated with different doses of IPP for thirty minutes, followed by a twenty minute period of incubation with 100 μM H₂O₂ to induce oxidative stress. Following induction of oxidative stress, DNA damage was quantified using the Comet assay, as described above. FIG. 17 graphically illustrates the attained DNA damage protection of A1AR-transfected HEK293 cells with isopentenyl diphosphate. As shown in FIG. 17, and when compared to the results shown in FIG. 10 that the same cells are resistant to 2CA-mediated antioxidative signaling, these cells responded readily to isopentenyl diphosphate.

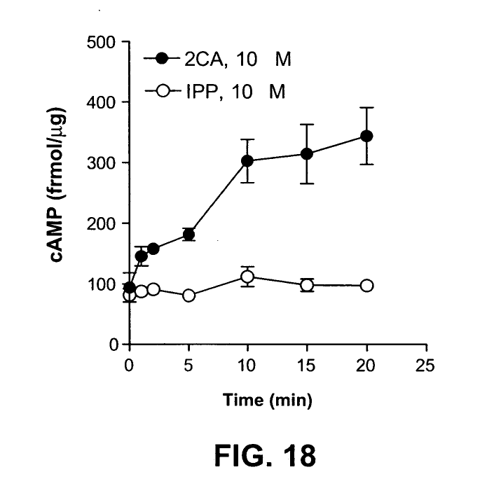

In another test, human fibroblastoid M1 cells also were stimulated with 10 μM 2-chloroadenosine or isopentenyl diphosphate, and cAMP levels were quantified. FIG. 18 graphically illustrates the effect of 2-chloroadenosine and isopentenyl diphosphate on c[AMP]ᵢ as a function of time. As shown in FIG. 18, while 2-chloroadenosine triggered an increase in c[AMP]₁ over time, whereas isopentenyl diphosphate did not trigger any meaningful increase in c[AMP]ᵢ.

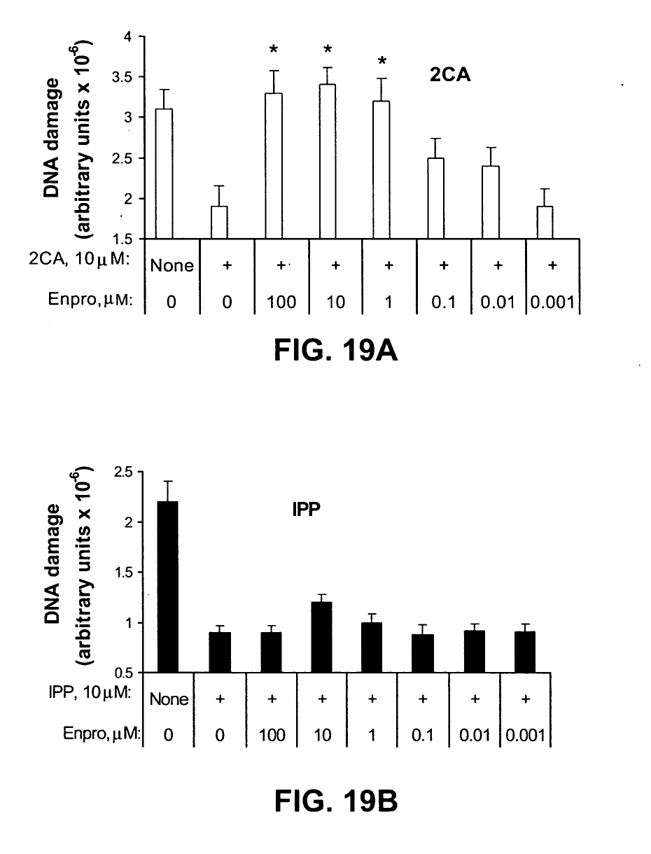

In yet another test, human fibroblastoid M1 cells were pre-incubated for fifteen minutes with different concentrations of the A2bAR antagonist, enprofylline (Enpro). These cells were then exposed to different doses of 2-chloroadenosine (FIG. 19A) or isopentenyl diphosphate (FIG. 19B) for thirty minutes, followed by incubation with 100 μM H₂O₂ for twenty minutes to induce oxidative stress. Following induction of oxidative stress, DNA damage was quantified using the Comet assay, as described above. On the y-axis of each of FIGS. 19A and 19B are DNA damage presented as arbitrary fluorescence intensity units. Based on the obtained results, it can be seen that unlike 2CA-mediated DNA damage protection, the IPP-mediated effect was resistant to the A2bAR antagonist, Enpro.

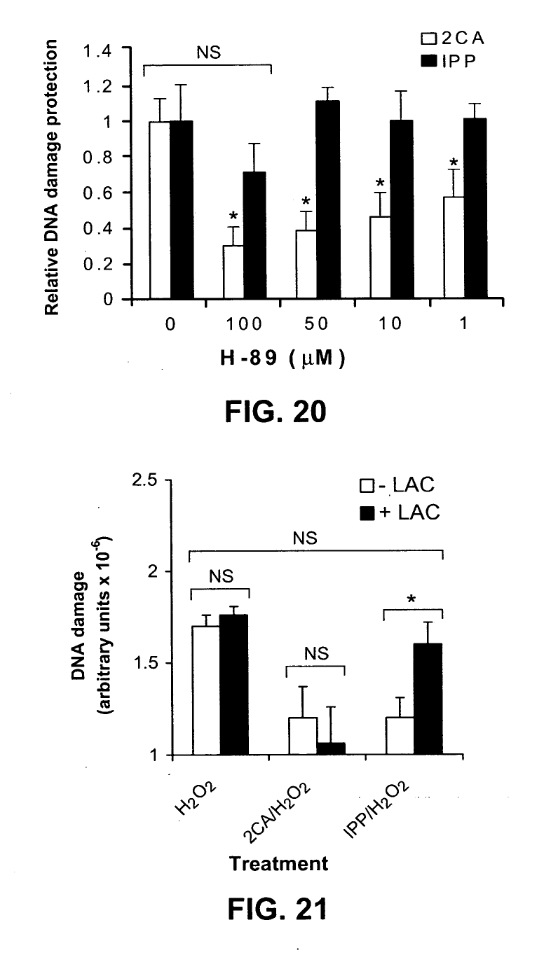

In still another test, human fibroblastoid M1 cells were pre-incubated for thirty minutes with different concentrations the PKA inhibitor, H-89. These cells were then exposed to 10 μM 2-chloroadenosine (FIG. 20, clear bars) or isopentenyl diphosphate (FIG. 20, shaded bars), followed by incubation with H₂O₂ to induce oxidative stress. Following induction of oxidative stress, DNA damage was quantified using the Comet assay, as described above. The obtained results are expressed as relative DNA damage protection, where maximum protection, in the absence of H-89, is defined as 1.0. Based on the obtained results, it can be seen that unlike 2CA-mediated DNA damage protection, the IPP-mediated effect was resistant to the PKA inhibitor, H-89. Thus, isopentenyl diphosphate signaling is independent of both the upstream and downstream segments of 2CA-mediated pathway,

IPP- and 2CA-mediated pathways could both be activated in a lineage- and species-independent manner. For example, murine L-cell fibroblasts, as well as human cells belonging to the lymphoid, fibroblastoid, myeloid, or neuronal lineages, all could respond to IPP- and 2CA-mediated antioxidative signaling (data not shown). In addition, the signaling reported herein is independent of p53 or DNA repair activity, as indicated by an intact DNA damage protection induction by both 2-chloroadenosine and isopentenyl diphosphate in p53-deficient cell lines HL60 and K562 and the nucleotide excision repair-deficient line XPA (data not shown). FIG. 21 graphically illustrates that antioxidative signaling by isopentenyl diphosphate—but not by 2-chloroadenosine—is dependent upon the proteasome-system.

More specifically, human fibroblastoid M1 cells were pre-incubated overnight with and without 5 μM of the proteasome inhibitor, lactacystin (LAC). These cells where then incubated with 10 μM 2-chloroadenosine or isopentenyl diphosphate, followed by incubation with 100 μM H₂O₂ for twenty minutes to induce oxidative stress. Following induction of oxidative stress, DNA damage was quantified using the Comet assay, as described above. Results are graphically shown in FIG. 21. Based on the results, it can be readily seen that lactacystin blocked IPP-mediated DNA damage protection signaling, whereas it did not similarly block 2CA-mediated DNA damage protection signaling. Thus, proteolytce cleavage of a putative downstream effector protein appears to be necessary for IPP-mediated signaling, but not for that of 2CA-mediated signaling.

Involvement of the Mevalonate Pathway in IPP-Mediated Antioxidative Signaling

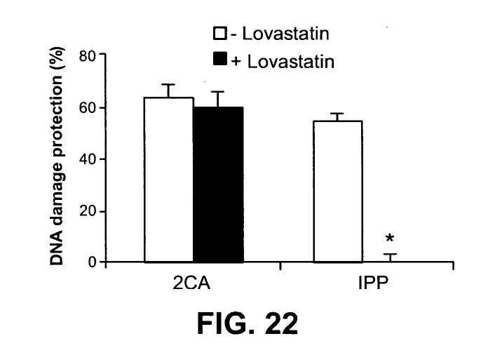

Isopentenyl diphosphate is an essential intermediate in the mevalonate pathway and is involved in isoprenylation of signal transduction proteins. A key regulatory enzyme in the mevalonate pathway is 3-hydroxy-3-methylglutaryl-coenzyme A (HMG-CoA) reductase. See FIG. 2, described above. To assess the role of the mevalonate pathway in IPP-mediated antioxidative signaling, DNA damage protecting activity of isopentenyl diphosphate in cells that had been pre-treated with lovastatin (an inhibitor of HMG-CoA reductase) was measured. Human fibroblastoid M1 cells were pre-incubated overnight with or without 5 μM lovastatin. These cells where then incubated with 10 μM 2-chloroadenosine or isopentenyl diphosphate, followed by incubation with 100 μM H₂O₂ for twenty minutes to induce oxidative stress. Following induction of oxidative stress, DNA damage was quantified using the Comet assay, as described above. The obtained results are graphically shown in FIG. 22. Based on the results, it can be readily seen that lovastatin completely blocked IPP-mediated DNA damage protection signaling, whereas it did not similarly block 2CA-mediated DNA damage protection signaling. Thus, the IPP-mediated DNA damage protection signaling—but, not 2CA-mediated DNA damage protection signaling—is dependent upon the mevalonate pathway.

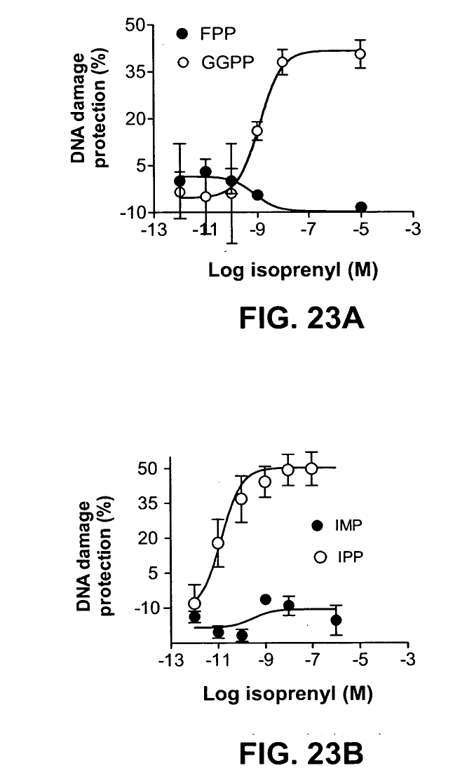

The structural specificity of isoprenoid-mediated antioxidative signaling also was examined by testing other isoprenyls, including the 5-carbon isopentenyl diphosphate variant, isopentenyl monophosphate (“IMP”), the 15-carbon isoprenyl compound, farnesyl diphosphate (“FPP”), and the 20-carbon isoprenyl compound, geranygeranyl diphosphate (“GGPP”). Human fibroblastoid M1 cells were incubated with various concentrations of different isoprenyls. These cells where then incubated with either FPP, GGPP, isopentenyl diphosphate (“IPP”), or isoprenyl monophosphate (“IMP”), followed by incubation with 100 μM H₂O₂ for twenty minutes to induce oxidative stress. Following induction of oxidative stress, DNA damage was quantified using the Comet assay, as described above. FIG. 23A graphically illustrates DNA damage protection obtainable with FPP or GGPP as a function of the logarithmic concentration of isoprenyl. FIG. 23A shows that GGPP was effective in triggering antioxidative activity, while FPP was completely devoid of such activity. FIG. 23B graphically illustrates DNA damage protection obtainable with IPP or IMP as a function of the logarithmic concentration of isoprenyl. Based on the obtained results, IPP and GGPP—but not IMP or FPP—triggered DNA damage-protection signaling. Furthermore, FIG. 23B shows that, despite its close structural similarity to isopentenyl diphosphate, isoprenyl monophosphate failed to trigger antioxidative signaling, indicating that the diphosphate moiety is appears to be responsible for the activity of isopentenyl diphosphate.

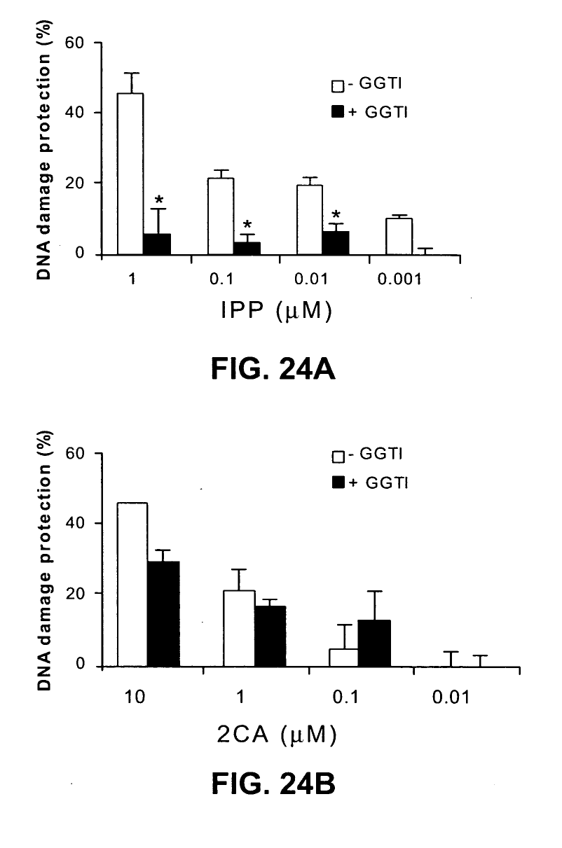

To determine if isoprenylated targets are involved in the IPP-mediated pathway, antioxidative signaling was studied in cells pre-treated with inhibitors of geranylgeranyl transferase (“GGT”) or farnesyl transferase (“FT”). Specifically, human fibroblastoid M1 cells were pre-incubated overnight with or without 0.2 μM of the GGT inhibitor, GGTI-2147. These cells where then incubated with either 2-chloroadenosine or isopentenyl diphosphate, followed by incubation with 100 μM H₂O₂ for twenty minutes to induce oxidative stress. Following induction of oxidative stress, DNA damage was quantified using the Comet assay, as described above. The obtained results are graphically shown in FIGS. 24A and 24B. Specifically, incubation of human fibroblastoid M1 cells overnight with 0.2 μM of the GGT inhibitor, GGTI-2147, effectively blocked IPP-mediated DNA damage protection.

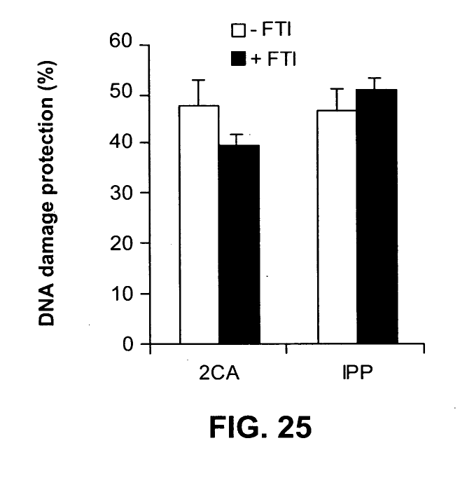

Similarly, human fibroblastoid M1 cells were pre-incubated overnight with or without 1.0 μM of the FT inhibitor, FTI-277. These cells where then incubated with either 10 μM 2-chloroadenosine or isopentenyl diphosphate, followed by incubation with 100 μM H₂O₂ for twenty minutes to induce oxidative stress. Following induction of oxidative stress, DNA damage was quantified using the Comet assay, as described above. The obtained results are graphically shown in FIG. 25. Specifically, overnight exposure to 1 μM of the FT inhibitor, FTI-277, had no effect on isopentenyl diphosphate activity. Neither GGTI-2147 (FIG. 24B) nor FTI-277 (FIG. 25) had a significant effect on 2CA-mediated DNA damage protection signaling. Taken together, these results indicate that a mevalonate pathway intermediate appears to be necessary for IPP-mediated DNA damage protection signaling—but not necessary for 2CA-mediated DNA damage protection signaling. Furthermore, and in contrast to the 2CA-mediated pathway, IPP-dependent DNA damage protection signaling involves a geranylgeranylated target.

The foregoing data suggest the involvement of the geranylgeranylated protein in the IPP-dependent antioxidative pathway. This suggestion is based on the findings that GGPP—but not FPP—reproduce the antioxidant effect of IPP and that pre-treatment of cells with the GGT inhibitor (GGTI-2147) blocked the antioxidant effect of IPP, while the FT inhibitor (FTI-277) had no similar blocking effect. Although the data indicate that a geranylgeranylated protein assists in IPP-triggered antioxidative signaling, the data do not make clear whether exogenously-added IPP or GGPP participate in de novo prenylation of proteins (protein prenylation is considered to be a stable modification). It is possible that active prenylation could occur within the short timeframe of (about thirty minutes of) exposure to isoprenyls. It is equally likely that IPP triggers rapid signaling events in which pre-existent, downstream geranylgeranylated proteins may play an effector role. The involvement of proteasome in IPP-mediated antioxidative signaling described herein is consistent with published literature. See H. Wu et al. (2002) FEBS Lett. 526:101-105; H. Kovacic et al. (2001) J. Biol. Chem. 276:45856-45861; M. Sundaresan et al. (1996) Biochem. J. 318:379-382; Y. Tanaka et al. (1995) Nature 375:155-158. Lactacystin has been previously shown to increase ROS levels by increasing the stability of Rac1. See id. Therefore, the differential role of the proteasome in IPP-mediated DNA damage protection signaling—but, not 2CA-mediated DNA damage protection signaling—appears to be a distinguishing feature between the two antioxidative pathways. Other differences are set forth in Table 2, below, wherein “+” indicates a dependence and “−” indicates independence:

TABLE 2

| | Mechanism of Signal Transduction | | | | |
|---|---|---|---|---|---|
| | | Mevalonate | | | |
| Agent | PKA | Pathway | GGT | FT | Proteasome |
| 2CA | + | − | − | − | − |
| IPP | − | + | + | − | + |

The methods disclosed herein provide numerous therapeutic benefits and utilize a heretofore unknown, potent antioxidant (isopentenyl diphosphate), having a simple chemical structure that is amenable to simple purification and/or synthesis. Furthermore, and contrary to currently leading antioxidants, isopentenyl diphosphate is not a vitamin regularly produced in the body and, therefore, isopentenyl diphosphate and pharmaceutical compositions comprising the same are less likely to cause allergic reactions and/or adverse effects.

The foregoing description is given for clearness of understanding only, and no unnecessary limitations should be understood therefrom, as modifications within the scope of the invention may be apparent to those having ordinary skill in the art.

1. A method of treating one or more oxidative stress-associated conditions, the method comprising administering to a host in need thereof a therapeutically effective amount of a pharmaceutical composition comprising isopentenyl diphosphate or a pharmaceutically acceptable salt thereof.

2. The method of claim 1, wherein the one or more oxidative stress-associated conditions is selected from the group consisting of diabetes mellitus, Down's Syndrome, exposure toxicity, gynecological diseases, inflammatory bowel disease, metabolic syndrome, pancreatitis, preeclampsia, prostate cancer, rheumatoid arthritis, systemic lupus erythematosus (SLE), and viral diseases.

3. The method of claim 2, wherein diabetes mellitus comprises IDDM and non-IDDM.

4. The method of claim 2, wherein exposure toxicity comprises toxicity experienced as a result of one or more of ionizing radiation, UV radiation, chemotherapeutic agents, genotoxic agents, and oxidative agents.

5. The method of claim 1, wherein the one or more oxidative stress-associated conditions is selected from the group consisting of diseases of the blood, brain/nervous system, breast, cardiovascular system, colon, gastrointestinal system, kidney, liver, respiratory system, skin, and stomach

6. The method of claim 5, wherein the diseases of the blood comprise acute lymphoblastic leukemia and Fanconi's anemia.

7. The method of claim 5, wherein cardiovascular diseases comprise atherosclerosis, hypertension, thrombosis, and heart disease.

8. The method of claim 7, wherein heart disease comprises coronary heart disease.

9. The method of claim 5, wherein the diseases of the brain/nervous system comprise Alzheimer's disease, amytrophic lateral sclerosis, cerebral amyloid angiopathy, Charcot Marie Tooth, dementia with Lewy bodies, Friedreich ataxia multiple sclerosis, and Parkinson's disease.

10. The method of claim 5, wherein the diseases of the breast comprise invasive ductal carcinoma and cancer.

11. The method of claim 5, wherein colon disease is colorectal cancer.

12. The method of claim 5, wherein diseases of the gastrointestinal system comprise inflammatory bowel disease.

13. The method of claim 5, wherein the diseases of the kidney comprise renal cell carcinoma and reperfusion injury.

14. The method of claim 5, wherein the diseases of the liver comprise chronic hepatitis, hepatitis C, hepatoblastoma, alcoholic liver disease, primary billiary cirrhosis, and heptacellular carcinoma.

15. The method of claim 5, wherein the diseases of the respiratory system comprise acute respiratory distress syndrome, asthma, chronic obstructive pulmonary dysfunction (COPD), cystic fibrosis, obstructive sleep apnea, squamous cell carcinoma, and, small cell carcinoma.

16. The method of claim 5, wherein the diseases of the skin comprise atopic dermatitis, skin neoplasma, skin wrinkling, pre-cancerous skin changes, viteligo, and psoriasis.

17. The method of claim 5, wherein the diseases of the stomach comprise H. pylori infection and cancer

18. The method of claim 1, wherein the one or more oxidative stress-associated conditions is selected from the group consisting of cancer and aging.

19. The method of claim 1, wherein the host is selected from the group consisting of a human beings, laboratory animals, pets, livestock, horses, and zoo specimens.

20. The method of claim 18, wherein the host is a human being.

21. The method of claim 1, wherein the composition is administered in an amount of about 0.1 to about 1000 mg/day.

22. The method of claim 1, wherein the composition is administered by oral, buccal, inhalation, sublingual, rectal, vaginal, intracisternal through lumbar puncture, transurethral, nasal, percutaneous, or parenteral administration.

23. The method of claim 22, wherein parenteral administration is by intravenous, intramuscular, subcutaneous, intracisternal or intracoronary administration.

24. The method of claim 22, wherein the composition is orally administered by aerosol sprays, capsules, dragees, gels, liquids, pills, sachets, slurries, suspensions, syrups, or tablets.

25. A method of preventing one or more oxidative stress-associated conditions, the method comprising administering to a host in need thereof a therapeutically effective amount of a pharmaceutical composition comprising isopentenyl diphosphate or a pharmaceutically acceptable salt thereof.

26. An article of manufacture comprising:
(a) a packaged pharmaceutical composition comprising isopentenyl diphosphate or a pharmaceutically acceptable salt thereof;
(b) an insert providing instructions for administration of the composition to treat or prevent an oxidative stress-associated condition in a host; and,
(c) a container for the packaged pharmaceutical composition and the insert.

27. A method of decreasing the oxidative stress level in a cell, the method comprising contacting the cell with isopentenyl diphosphate or a pharmaceutically acceptable salt thereof under conditions effective to decrease the level of an oxidizing species present in the cell in response to an oxidative stress compared to the same conditions when the isopentenyl diphosphate or a pharmaceutically acceptable salt thereof is not present.

28. A method of decreasing the oxidative stress level in a host, the method comprising administering to the host isopentenyl diphosphate or a pharmaceutically acceptable salt thereof under conditions effective to decrease the level of an oxidizing species present in the host in response to an oxidative stress compared to the same conditions when the isopentenyl diphosphate or a pharmaceutically acceptable salt thereof is not present.

29. A method of treating a biomolecule damaged by oxidative stress, the method comprising the step of administering isopentenyl diphosphate or a pharmaceutically acceptable salt thereof to the biomolecule.

30. The method of claim 29, wherein the biomolecule is selected from the group consisting of proteins, lipids, and nucleic acids.

31. A method of preventing oxidative stress-associated damage to a biomolecule, the method comprising administering isopentenyl diphosphate or a pharmaceutically acceptable salt thereof to the biomolecule.

32. The method of claim 31, wherein the biomolecule is selected from the group consisting of proteins, lipids, and nucleic acids.

33. A pharmaceutical composition comprising isopentenyl diphosphate or a pharmaceutically acceptable salt thereof, and a pharmaceutically acceptable carrier.

34. The composition of claim 33, further comprising an ingredient selected from the group consisting of absorbents, abrasives, anti-acne agents, anticaking agents, antifoaming agents, antimicrobial agents, antioxidants other than isopentenyl diphosphate, binders, biological additives, buffering agents, bulking agents, chelating agents, chemical additives, colorants, cosmetic astringents, cosmetic biocides, denaturants, drug astringents, emulsifiers, external analgesics, film formers, foam boosters, fragrance comoponents, humectants, hydrotropes, opacifying agents, pH adjusters, plasticers, preservatives, propellants, reducing agents, sequestrants, skin bleaching agents, skin-conditioning agents, skin protectants, skin sensates, solvents, solubilizing agents, suspending agents, sunscreen agents, ultraviolet light absorbers, and viscosity increasing agents.
